SEQUENCE LISTING (39 sequences)

SEQ ID NO: 1 - DNA, 48 bp, Artificial sequence
  synthetic
atgggannknnknnknnknnknnknnknnknnknnkggggggcccccc

SEQ ID NO: 2 - protein, 16 aa, Artificial sequence
  misc_feature (3)..(3): The ′Xaa′ at location 3 stands for Lys, Asn, Arg, Ser, Thr, Met, Ile, Glu, Asp, Gly, Ala, Val, Gln, His, Pro, Leu, Tyr, Trp, Cys, or Phe.
MGXXXXXXXXXXGGPP

SEQ ID NO: 3 - protein, 61 aa, Artificial sequence
  coiled-coil presentation structure
MGCAALESEVSALESEVASLESEVAALGRGDMPLAAVKSKLSAVKSKLASVKSKLAACGPP

SEQ ID NO: 4 - protein, 69 aa, Artificial sequence
  minibody presentation structure
MGRNSQATSGFTFSHFYMEWVRGGEYIAASRHKHNKYTTEYSASVKGRYIVSRDTSQSILYLQKKKGPP

SEQ ID NO: 5 - protein, 7 aa, Simian virus 40
PKKKRKV

SEQ ID NO: 6 - protein, 6 aa, Homo sapiens
ARRRRP

SEQ ID NO: 7 - protein, 10 aa, Mus musculus
EEVQRKRQKL

SEQ ID NO: 8 - protein, 9 aa, Mus musculus
EEKRKRTYE

SEQ ID NO: 9 - protein, 20 aa, Xenopus laevis
AVKRPAATKKAGQAKKKKLD

SEQ ID NO: 10 - protein, 31 aa, Mus musculus
MASPLTRFLSLNLLLLGESILGSGEAKPQAP

SEQ ID NO: 11 - protein, 21 aa, Homo sapiens
MSSFGYRTLTVALFTLICCPG

SEQ ID NO: 12 - protein, 51 aa, Mus musculus
PQRPEDCRPRGSVKGTGLDFACDIYIWAPLAGICVALLLSLIITLICYHSR

SEQ ID NO: 13 - protein, 33 aa, Homo sapiens
MVIIVTVVSVLLSLFVTSVLLCFIFGQHLRQQR

SEQ ID NO: 14 - protein, 37 aa, Rattus sp.
PNKGSGTTSGTTRLLSGHTCFTLTGLLGTLVTMGLLT

SEQ ID NO: 15 - protein, 14 aa, Gallus gallus
MGSSKSKPKDPSQR

SEQ ID NO: 16 - protein, 26 aa, Homo sapiens
LLQRLFSRQDCCGNCSDSEEELPTRL

SEQ ID NO: 17 - protein, 20 aa, Rattus norvegicus
KQFRNCMLTSLCCGKNPLGD

SEQ ID NO: 18 - protein, 19 aa, Homo sapiens
LNPPDESGPGCMSCKCVLS

SEQ ID NO: 19 - protein, 5 aa, Artificial sequence
  lysosomal degradation sequence
KFERQ

SEQ ID NO: 20 - protein, 36 aa, Cricetulus griseus
MLIPIAGFFALAGLVLIVLIAYLIGRKRSHAGYQTI

SEQ ID NO: 21 - protein, 35 aa, Homo sapiens
LVPIAVGAALAGVLILVLLAYFIGLKHHHAGYEQF

SEQ ID NO: 22 - protein, 27 aa, Saccharomyces cerevisiae
MLRTSSLFTRRVQPSLFSRNILRLQST

SEQ ID NO: 23 - protein, 25 aa, Saccharomyces cerevisiae
MLSLRQSIRFFKPATRTLCSSRYLL

SEQ ID NO: 24 - protein, 64 aa, Saccharomyces cerevisiae
MFSMLSKRWAQRTLSKSFYSTATGAASKSGKLTQKLVTAGVAAAGITASTLLYADSLTAEAMTA

SEQ ID NO: 25 - protein, 41 aa, Saccharomyces cerevisiae
MKSFITRNKTAILATVAATGTAIGAYYYYNQLQQQQQRGKK

SEQ ID NO: 26 - protein, 4 aa, Homo sapiens
KDEL

SEQ ID NO: 27 - protein, 15 aa, unidentified adenovirus
LYLSRRSFIDEKKMP

SEQ ID NO: 28 - protein, 15 aa, Homo sapiens
LTEPTQPTRNQCCSN

SEQ ID NO: 29 - protein, 9 aa, Unknown
  cyclin B1 destruction sequence
RTALGDIGN

SEQ ID NO: 30 - protein, 20 aa, Unknown
  signal sequence from Interleukin-2
MYRMQLLSCIALSLALVTNS

SEQ ID NO: 31 - protein, 29 aa, Homo sapiens
MATGSRTSLLLAFGLLCLPWLQEGSAFPT

SEQ ID NO: 32 - protein, 27 aa, Homo sapiens
MALWMRLLPLLALLALWGPDPAAAFVN

SEQ ID NO: 33 - protein, 18 aa, Influenza virus
MKAKLLVLLYAFVAGDQI

SEQ ID NO: 34 - protein, 24 aa, Unknown
  signal sequence from Interleukin-4
MGLTSQLLPPLFFLLACAGNFVHG

SEQ ID NO: 35 - protein, 10 aa, Artificial sequence
  stability sequence
MGXXXXGGPP

SEQ ID NO: 36 - protein, 5 aa, Artificial sequence
  linker consensus sequence
GSGGS

SEQ ID NO: 37 - protein, 4 aa, Artificial sequence
  linker consensus sequence
GGGS

SEQ ID NO: 38 - protein, 20 aa, Artificial sequence
  consensus sequence for SH-3 domain binding protein
MGXXXXXRPLPPXPXXGGPP

SEQ ID NO: 39 - DNA, 63 bp, Artificial sequence
  Oligonucleotide consensus sequence for SH-3 domain binding protein
atgggcnnknnknnknnknnkagacctctgcctccasbkgggsbksbkggaggcccaccttaa

(19) United States
(12) Patent Application Publication
(10) Pub. No.: US 2003/0219723 A1
(43) Pub. Date: Nov. 27, 2003

(54) Compositions and methods for screening and identifying anti-HCV agents

(76) Inventors: Henry H. Lu, Foster City, CA (US); Peiyong Huang, Sunnyvale, CA (US); Todd Kinsella, Joliet, IL (US); Anthony Martinez, San Francisco, CA (US)

Correspondence Address: DORSEY & WHITNEY LLP, INTELLECTUAL PROPERTY DEPARTMENT, 4 EMBARCADERO CENTER, SUITE 3400, SAN FRANCISCO, CA, 94111, US

(21) Appl. No.: 10/152,163

(22) Filed: May 20, 2002

(51) Int. Cl. C12Q 1/70; C07H 21/04

(52) U.S. Cl. 435/5; 536/23.72

(57) ABSTRACT

The field of the invention is methods for screening for effector peptides, polypeptides and fragments thereof and RNA molecules selected inside living cells that have anti-HCV activity.

### FIELD OF THE INVENTION

The field of the invention is methods for screening for effector peptides, polypeptides and fragments thereof and RNA molecules selected inside living cells that have anti-HCV activity.

### BACKGROUND OF THE INVENTION

Hepatitis C virus (HCV) infection is an important clinical problem worldwide. In the United States alone, an estimated four million individuals are chronically infected with HCV. HCV, the major etiologic agent of non-A, non-B hepatitis, is transmitted primarily by transfusion of infected blood and blood products (Cuthbert et al., 1994, Clin. Microbiol. Rev. 7:505-532; Mansell et al., 1995, Semin. Liver Dis. 15:15-32). Prior to the introduction of anti-HCV screening in mid-1990, HCV accounted for 80-90% of posttransfusion hepatitis cases in the United States. Currently, injection drug use is probably the most common risk factor for HCV infection, with approximately 80% of this population seropositive for HCV. A high rate of HCV infection is also seen in individuals with bleeding disorders or chronic renal failure, groups that have frequent exposure to blood and blood products.

Acute infection with HCV results in persistent viral replication and progression to chronic hepatitis in approximately 90% of cases. For many patients, chronic HCV infection results in progressive liver damage and the development of cirrhosis. In patients with an aggressive infection, cirrhosis can develop in as little as two years, although a time span of 10-20 years is more typical. In 30-50% of chronic HCV patients, liver damage may progress to the development of hepatocellular carcinoma. In general, hepatocellular carcinoma is a late occurrence and may take greater than 30 years to develop (Bisceglie et al., 1995, Semin. Liver Dis. 15:64-69). The relative contribution of viral or host factors in determining disease progression is not clear.

HCV is an enveloped virus containing a positive-sense single-stranded RNA genome of approximately 9.5 kb. On the basis of its genome organization and virion properties, HCV has been classified as a separate genus in the family Flaviviridae, a family that also includes pestiviruses and flaviviruses (Alter, 1995, Semin. Liver Dis. 15:5-14). The viral genome consists of a lengthy 5′ untranslated region (UTR), a long open reading frame encoding a polyprotein precursor of approximately 3011 amino acids, and a short 3′ UTR. The 5′ UTR is the most highly conserved part of the HCV genome and is important for the initiation and control of polyprotein translation.

Translation of the HCV genome is initiated by a cap-independent mechanism known as internal ribosome entry. This mechanism involves the binding of ribosomes to an RNA sequence known as the internal ribosome entry site (IRES)(reviewed in Sonenberg & Meerovitch, 1990, which is incorporated herein by reference). As their names imply, these are sequences which enable ribosomes to bind to viral RNAs at internal sites rather than at the 5′-ends of these RNAs; having bound, the ribosomes can then migrate to the AUG initiator codon and begin translation. An RNA pseudoknot structure has recently been determined to be an essential structural element of the HCV IRES. As such, the IRES regulatory element is an essential component of viral translation and replication.

It is thought that other viruses also utilize an IRES-type translation regulatory system. That is, It is thought that other viruses employ nucleic acid sequences responsible for preferential translation of viral RNAs. Viruses whose RNAs are believed to be preferentially translated because of specific viral nucleic acid sequences currently include picornaviruses, hepatitis B virus, hepatitis C virus, influenza virus, adenovirus and cytomegalovirus.

Picornaviruses are an important class of viruses responsible for a broad array of human and animal diseases (reviewed in Chapters 20-23 in Fields B. N., Knipe D. M. (eds): Fields Virology, ed. 2, Raven Press, New York, 1990). They include polioviruses, rhinoviruses (the most frequent cause of respiratory tract infections), coxsackie viruses (a cause of gastrointestinal illnesses, myocarditis and meningitis), hepatitis A virus, and foot-and-mouth disease viruses. Picornaviruses are single-stranded RNA viruses whose RNA genomes are positive-sense and nonsegmented. The genomic RNA strand inside each virus is translated when the virus enters a host cell. One of the proteins translated from the incoming RNA genome is an RNA-dependent RNA polymerase which copies the viral genome to produce additional full-length viral RNAs. Some off these RNAs are translated to produce additional viral proteins, and some are packaged as RNA genomes into a new generation of viruses. Each RNA is translated into a single “polyprotein” which is cleaved as it is translated to yield individual viral proteins.

Hepatitis B virus is a hepatovirus which can cause severe liver disease and which is very widespread (reviewed in chapter 78 of Fields B. N., Knipe D. M. (eds): Fields Virology, ed. 2, Raven Press, New York, 1990). The virus has a very unusual genome and an equally unusual method of replication. In brief, the viral genome consists of partially double-stranded DNA. The negative-sense strand is a full circle, but the two ends of this circle are not covalently joined. The positive-sense strand is incomplete and its length is not the same in all molecules, so that the single-stranded region of the genome varies in length from approximately 15%-60% of the circle length in different molecules. When the virus infects a cell, the infecting genome appears to be converted to closed circular (cc) viral DNA which can be detected in the cell nucleus. This DNA is transcribed into (positive-sense) viral mRNAs, one of which encodes a reverse transcriptase which makes negative-sense DNA copies of viral RNA to produce further viral genomes. The (incomplete) positive-.sense DNA strand of the genome is produced by partial copying of the negative-sense strand, with synthesis primed by a short viral oligoribonucleotide. The viral reverse transcriptase (P protein) is encoded within a long mRNA which also includes the coding sequence for the major viral core protein (C protein). The C-protein sequence is upstream of the P-protein sequence in the mRNA and partially overlaps it, in a different reading frame. Data from gene fusions which place a reporter gene downstream of the C-P overlap region suggest that translation of the P protein involves initiation at an internal ribosome entry site within the C-protein coding sequence (Chang et al., (1990), Proc. Natl. Acad. Sci. USA 87, 5158-5162). This interpretation is supported by the observation that defined fragments of the C-protein sequence increase translation of the downstream cistron when placed between the two cistrons of a dicistronic mRNA or in the 5.mu.-UTR of a monocistronic mRNA (Jean-Jean et al., (1989) J. Virology 63, 5451 5454). Thus, the ability to translate a crucial viral protein is highly dependent upon the presence of a specific viral nucleic acid sequence translationally linked to the coding sequence. Thus, viral translational regulatory sequences are attractive targets for therapeutics because they are important mediators of viral translation and subsequent propagation and viability.

Historically, regulatory pathways or other signal transduction pathways have been analyzed by biochemistry or genetics. The biochemical approach dissects a pathway in a “stepping-stone” fashion: find a molecule that acts at, or is involved in, one end of the pathway, isolate assayable quantities and then try to determine the next molecule in the pathway, either upstream or downstream of the isolated one. The genetic approach is classically a “shot in the dark”: induce or derive mutants in a signaling pathway and map the locus by genetic crosses or complement the mutation with a cDNA library. Limitations of biochemical approaches include a reliance on a significant amount of pre-existing knowledge about the constituents under study and the need to carry such studies out in vitro, post-mortem. Limitations of purely genetic approaches include the need to first derive and then characterize the pathway before proceeding with identifying and cloning the gene.

Screening molecular libraries of chemical compounds for drugs that regulate signal systems has led to important discoveries of great clinical significance. Cyclosporin A (CsA) and FK506, for examples, were selected in standard pharmaceutical screens for inhibition of T-cell activation. It is noteworthy that while these two drugs bind completely different cellular proteins—cyclophilin and FK506 binding protein (FKBP), respectively, the effect of either drug is virtually the same—profound and specific suppression of T-cell activation, phenotypically observable in T cells as inhibition of mRNA production dependent on transcription factors such as NF-AT and NF-KB. Libraries of small peptides have also been successfully screened in vitro in assays for bioactivity. The literature is replete with examples of small peptides capable of modulating a wide variety of signaling pathways. For example, a peptide derived from the HIV-1 envelope protein has been shown to block the action of cellular calmodulin.

A major limitation of conventional in vitro screens is delivery. While only minute amounts of an agent may be necessary to modulate a particular cellular response, delivering such an amount to the requisite subcellular location necessitates exposing the target cell or system to relatively massive concentrations of the agent. The effect of such concentrations may well mask or preclude the targeted response.

Accordingly, there exists a need for effective strategies to target a viral IRES in an in vivo setting to establish a method of inhibiting replication or production of the virus. In addition, there exists a need to establish a method of screening for target molecules that inhibit or interfere with the virus by inhibiting the IRES.

### SUMMARY OF THE INVENTION

In accordance with the objects outlined above, the present invention provides a method of assaying for a potential anti-HCV agent comprising providing cells comprising a nucleic acid construct comprising an HCV internal ribosome entry site (IRES), and a reporter gene. The method further includes contacting the cells with a library of nucleic acids, whereby the nucleic acids are expressed in the cells forming candidate agents, and screening the cells for altered expression of the reporter gene.

In addition the invention provides a method for assaying for anti-HCV agents comprising providing cells comprising the HCV genome, contacting the cells with a library of nucleic acids, whereby the nucleic acids are expressed in the cells forming candidate agents and screening for cells exhibiting altered HCV production.

In addition the invention provides a method for assaying for anti-HCV agents comprising providing Hepatitis viral particle packaging cells comprising a nucleic acid comprising a replication incompetent HCV genome, harvesting virus therefrom, infecting a second population of cells with the virus, contacting the second population of cells with a library of nucleic acids, whereby the nucleic acids are expressed in the cells forming candidate agents and screening for cells exhibiting an altered phenotype, wherein the altered phenotype is an indication of anti-HCV activity induced by the expression product.

In addition the invention provides a method of assaying for a potential antiviral agent comprising providing cells comprising a nucleic acid construct comprising a viral internal ribosome entry site (IRES) and a reporter gene, contacting the cells with a library of nucleic acids, whereby the nucleic acids are expressed in the cells forming candidate agents, and screening the cells for altered expression of the reporter gene.

In addition the invention provides a method of assaying for a potential antiviral agent comprising providing cells comprising a nucleic acid construct comprising a viral internal ribosome entry site (IRES) and a reporter gene, contacting the cells with a retroviral library of nucleic acids, whereby the nucleic acids are expressed in the cells forming candidate agents, and screening the cells for altered expression of the reporter gene.

In addition the invention provides a cell culture comprising a plurality of eukaryotic cells comprising a first nucleic acid construct comprising a viral IRES and a reporter gene and a second nucleic acid construct comprising nucleic acid encoding a candidate agent, wherein each of the plurality of eukaryotic cells comprises the first construct and a different second construct.

### BRIEF DESCRIPTION OF THE DRAWINGS

FIG. 1 Creation of a library of random peptides in a retrovirus DNA construct by PCR.

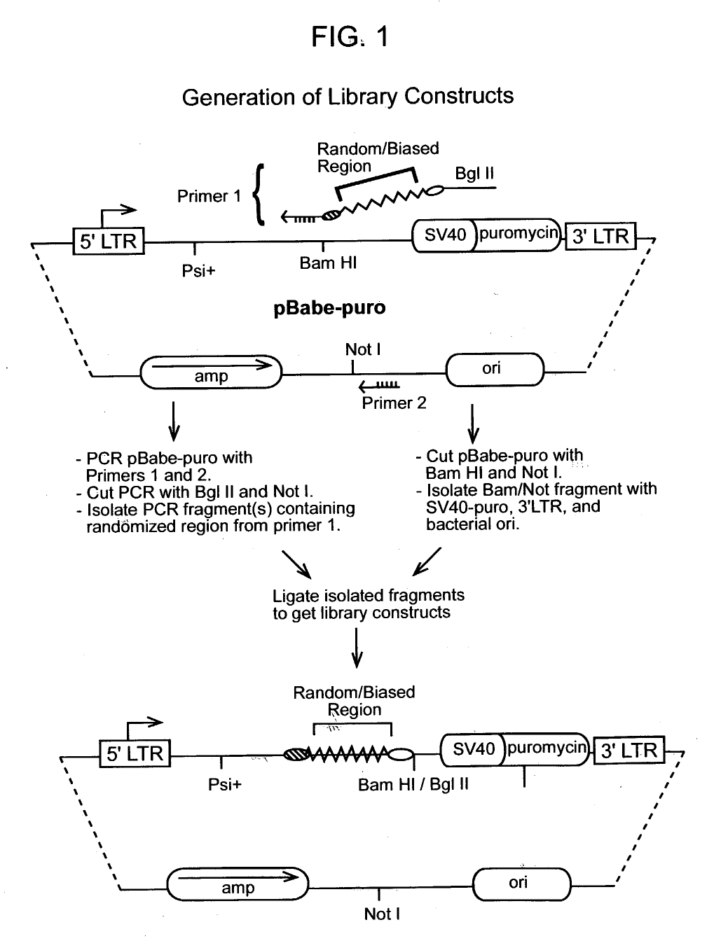

FIG. 2 Creation of a library of random peptides in a retrovirus DNA construct by primed DNA synthesis.

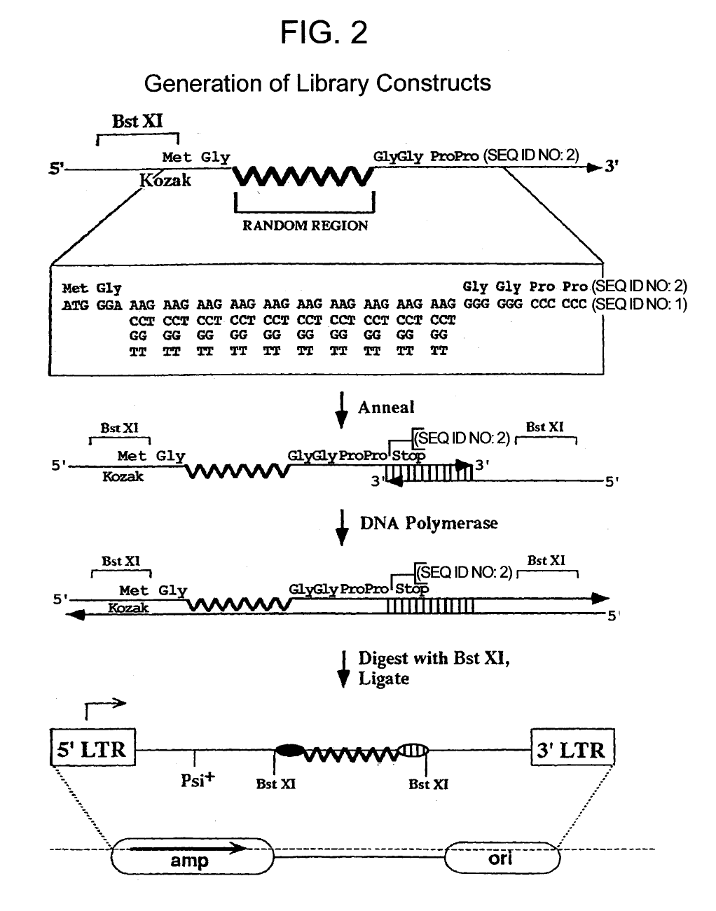

FIG. 3 Presentation constructs for localizing presentation structures to specific cellular locales.

FIG. 4 Schematic of a retroviral construct.

FIG. 5 An HCV IRES screen cell line expressing dual color reporters. An HCV-IRES specific inhibitor will block the expression of IRES-driven GFP. A. Negative control with no reporter. B.

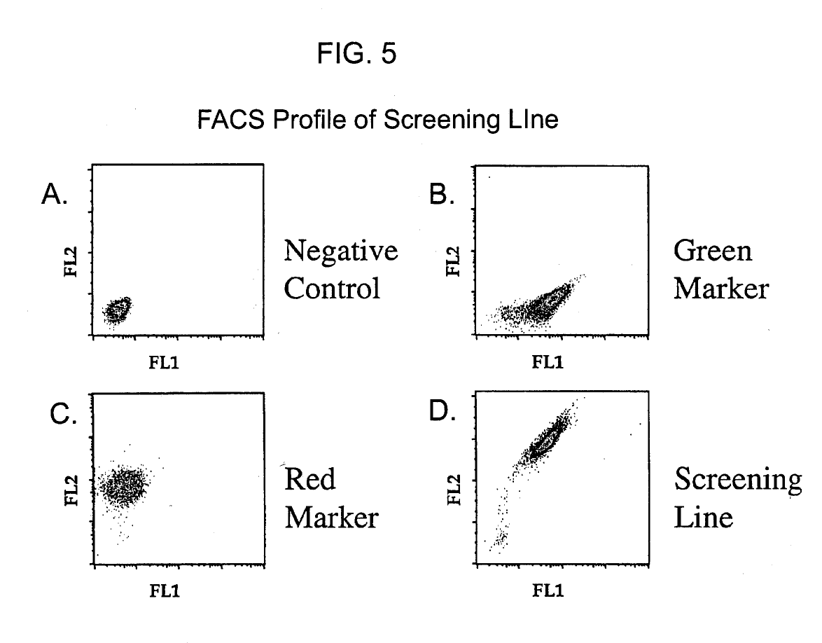

Detection of Green Marker. C. Detection of Red Marker. D. Detection of fluorescence profile of Screening Cell Line.

FIG. 6A. Schematic of dual marker construct. Cap-dependent RFP coding sequence is followed by GFP coding sequence. GFP coding sequence is under the control of HCV IRES. B. Depicts FACS profile of cells expressing the dual reporter in the absence of IRES specific inhibition. This screen cell line expresses both cap-dependent RFP and HCV-IRES driven GFP genes. C. Depicts FACS profile of cells expressing the dual reporter in the presence of IRES specific inhibition. Green fluorescence is diminished while Red fluorescence is not.

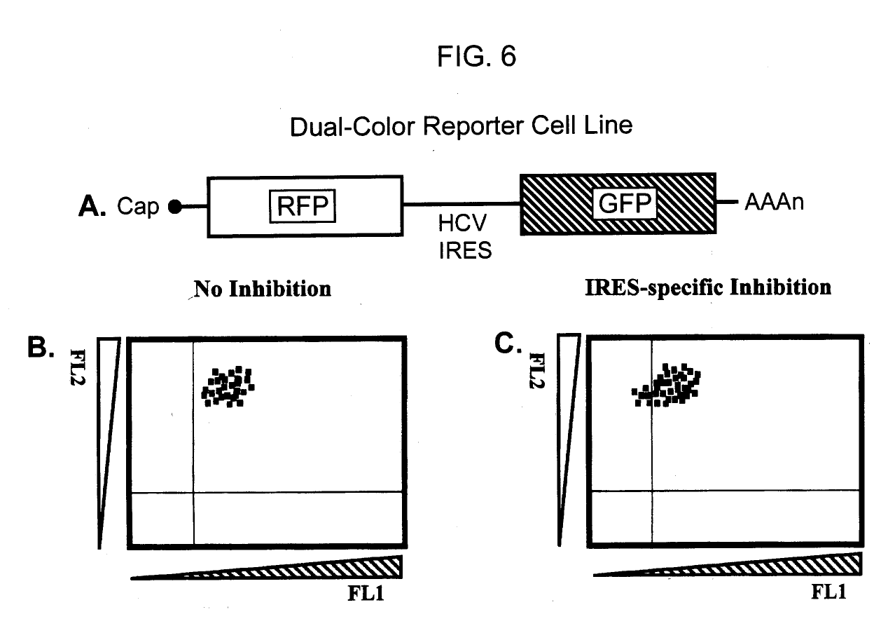

FIG. 7 Depicts constructs for cyclic peptide screening against HCV IRES using a tri-fluorescence screen. A. Depicts the construct for the screening cell line that includes Rluc followed by GFP; GFP is under the control of the HCV IRES. B. Depicts the construct for the cyclic peptide library of 5-mers. The construct, which provides for inducible expression, includes the intein C-terminus-Ser-4 variable amino acids-intein N-terminus fused to BFP.

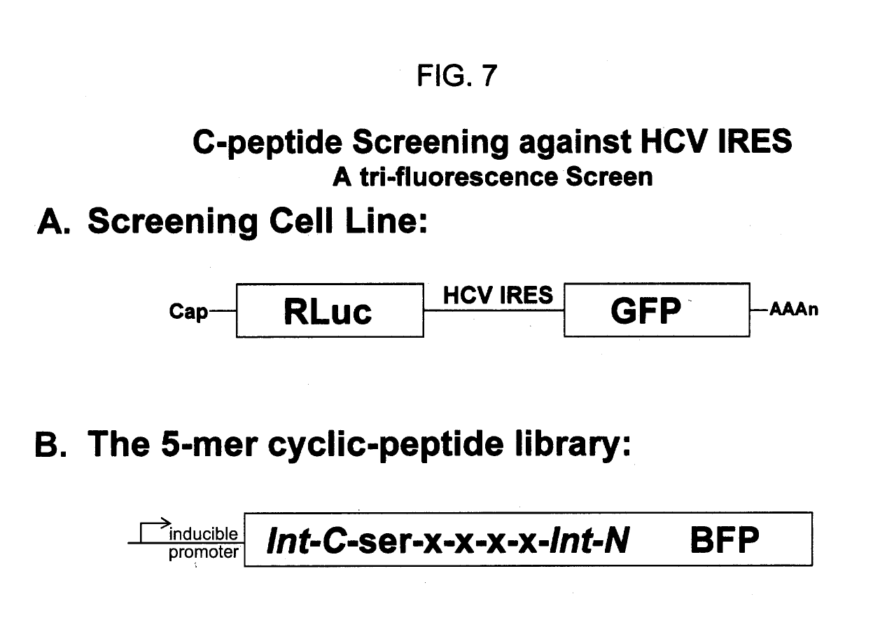

FIG. 8 Depicts DOX regulation of BFP-fused cyclic peptide expression in the screen cell line. BFP-cyclic peptide expression was inhibited by addition of Dox. Upon removing DOX, BFP-cyclic peptide was induced as shown by FACS analysis.

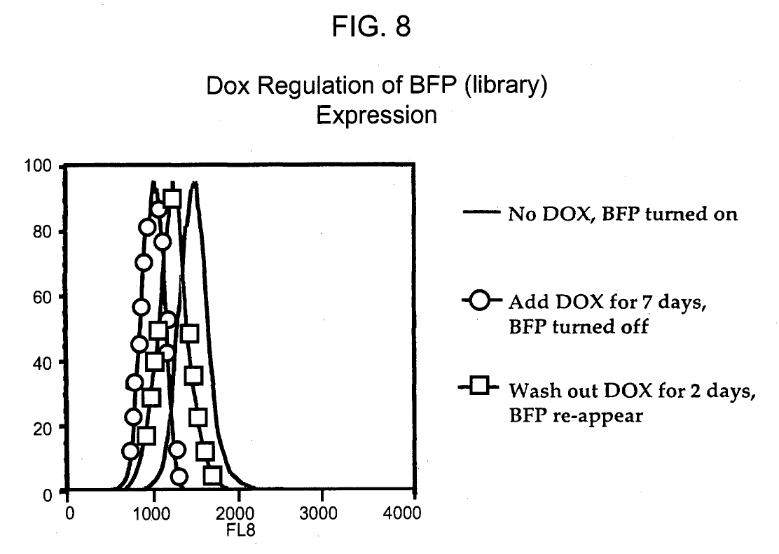

FIG. 9 Demonstrates GFP fluorescence in the presence or absence of positive cyclic peptide hits that emerged from a 5-mer cyclic peptide library screen. A and B depict different positives identified in the screen. In each case, expression of the “hit” (Library On) results in reduced GFP signal.

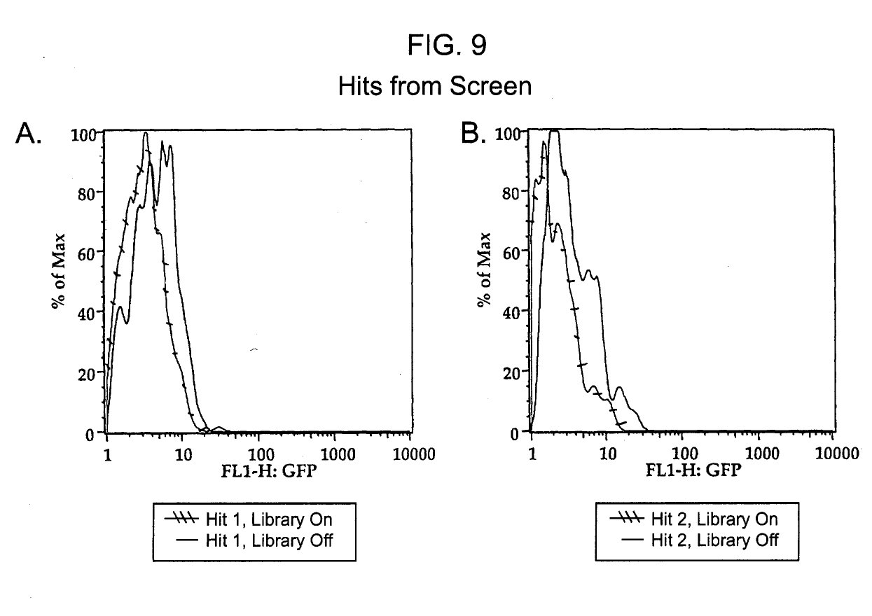

FIG. 10 Demonstrates GFP fluorescence in the presence or absence of Ribozyme targeted to the HCV IRES.A. Depicts GFP fluorescence in the presence of active Ribozyme. B. Depicts GFP fluorescence in the presence of inactive ribozyme.

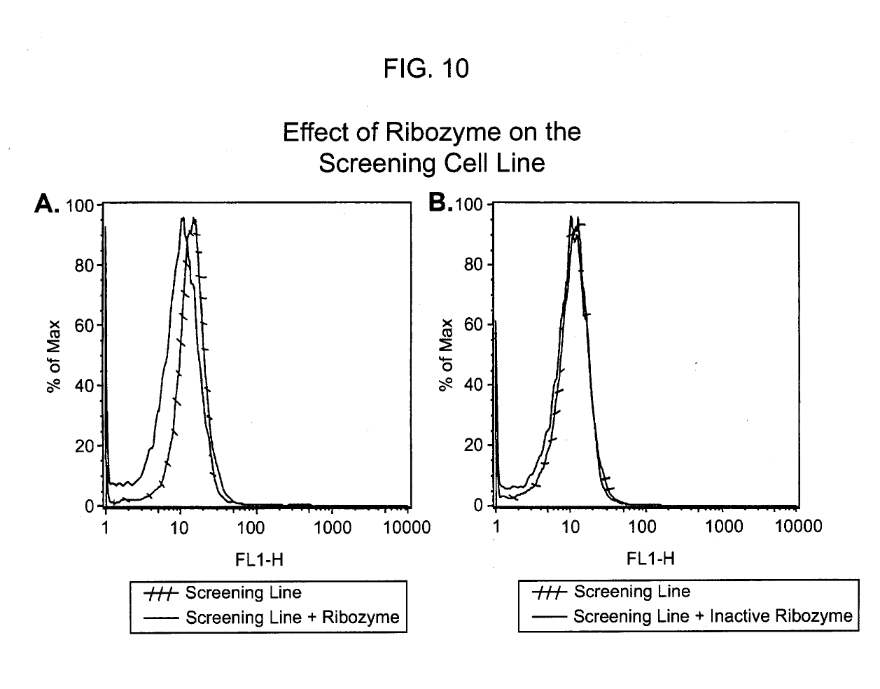

### DETAILED DESCRIPTION OF THE INVENTION

The present invention provides methods and compositions to create, effectively introduce into cells and screen compounds that affect a viral viability or affect viral production. In some embodiments, little or no knowledge of the virus is required, other than a presumed signaling event and an observable physiologic change in the target or host cell. However, in a preferred embodiment particular viral regulatory pathways are targeted. The disclosed methods are conceptually distinct from prior library search methods in that it is an in vivo stratagem for accessing viral signaling mechanisms. The invention also provides for the isolation of the constituents of the pathway, the tools to characterize the pathway, and lead compounds for pharmaceutical development.

The present invention provides methods for the screening of candidate bioactive agents which are capable of altering the phenotype of cells infected with a virus or containing a viral reporter system. The methods of the present invention provide a significant improvement over conventional screening techniques, as they allow the rapid screening of large numbers of random oligonucleotides and/or their corresponding expression products in a single, in vivo step. Thus, by delivering the random oligonucleotides to cells and screening the same cells, without the need to collect or synthesize in vitro the candidate agents, highly efficient screening is accomplished.

Thus, the present invention provides methods for screening candidate bioactive agents for a bioactive agent capable of altering the phenotype of a cell that is infected with a virus or contains a viral reporter system.

By “candidate bioactive agents” or “candidate drugs” or “candidate expression products” or grammatical equivalents herein is meant the expression product of a candidate nucleic acid which may be tested for the ability to ly alter the phenotype of a cell. As is described below, the candidate bioactive agents are the expression products of candidate nucleic acids, and encompass several chemical classes, including peptides and nucleic acids such as DNA, messenger RNA (mRNA), antisense RNA, ribozyme components, etc. Thus, the candidate bioactive agents (expression products) may be either translation products of the candidate nucleic acids, i.e. peptides, or transcription products of the candidate nucleic acids, i.e. either DNA or RNA.

In a preferred embodiment, the candidate bioactive agents are translation products of the candidate nucleic acids. In this embodiment, the candidate nucleic acids are introduced into the cells, and the cells express the nucleic acids to form peptides. Thus, in this embodiment, the candidate bioactive agents are peptides. Generally, peptides ranging from about 4 amino acids in length to about 100 amino acids may be used, with peptides ranging from about 5 to about 50 being preferred, with from about 5 to about 30 being particularly preferred and from about 6 to about 20 being especially preferred.

In a preferred embodiment the candidate bioactive agents are dominant negative protein fragments.

That is, the candidate expression products are the products of truncated cDNA clones that encode truncated polypeptides. The truncated polypeptides, when expressed, act as dominant negative molecules inhibiting the target with which they associate, or preventing association of the target with another molecule. In this embodiment the candidate nucleic acids, which include truncated nucleic acids, are introduced into cells and the cells express the nucleic acids forming truncated polypeptides. In a preferred embodiment the truncated cDNA fragments are randomly generated fragments, i.e. from a randomly fragmented cDNA library. The truncated polypeptides range from about 20 amino acids to 1000 amino acids in length, with from about 50 to 500 being preferred, with from about 100 to about 400 being particularly preferred and from about 150 to about 250 amino acids being especially preferred.

In a preferred embodiment the candidate bioactive agents may not necessarily be dominant negative protein fragments. Rather the candidate bioactive agents are the expression product of cDNA clones that have frame shift mutations which result in the generation of random expression products. While these may function as dominant negatives, they are not necessarily designed for a particular target.

In a preferred embodiment, the candidate bioactive agents are transcription products of the candidate nucleic acids, and are thus also nucleic acids. The transcription products may be either primary transcripts or secondary translation products. That is, using the retroviral reverse transcriptase, primary DNA is made which are later converted into double stranded DNA. Additionally, using the primary DNA, RNA transcripts can be generated within the cell, including mRNA, antisense RNA and ribozymes or portions thereof.

In a preferred embodiment the candidate nucleic acids are inserted into the expression vector in an inverted orientation. Thus, the transcription product functions as an antisense molecule. In this embodiment, this “flipped cDNA” insert serves as a template for transcription of an RNA species, an antisense RNA, that inhibits the target. By “flipped cDNA” is meant that the cDNA insert of the library is inserted in the vector in the opposite or inverted orientation relative to the orientation that encodes a polypeptide.

In a preferred embodiment the candidate nucleic acids are small interfering RNA (mRNA) molecules.

These molecules suppress gene expression through RNA interference. RNA interference is summarized in Science vol. 292, 25 May 2002, pp. 1469-1471, Nature, 411, 494 (2001) and Science vol. 296, 19 April 2002, pp 550-553, all of which are expressly incorporated herein by reference.

At a minimum, the candidate bioactive agents comprise randomized expression products of the candidate nucleic acids. That is, every candidate bioactive agent has a randomized portion, as defined below, that is the basis of the screening methods outlined herein. In addition, to the randomized portion, the candidate bioactive agent may also include a fusion partner.

In a preferred embodiment is a cyclic peptide. That is, the bioactive agent is a peptide, preferably random, that is joined at its N- and C-termini. However, in some embodiments it is preferable to have biased positions in the library. That is, some positions within the variable region of the library are fixed or limited to only particular amino acids.

Libraries designed to form cyclic peptides are described in more detail in U.S. Ser. No. 09/800,770, filed Mar. 6, 2001, and WO 00/36093, both of which are expressly incorporated herein by reference in their entirety. In this embodiment, generally, a candidate nucleic acid is inserted into a vector between sequences encoding the N and C termini of an intein. Upon expression, the intein forms a circular structure and ligates the N and C terminus of the expression product of the candidate nucleic acid, thereby forming a cyclic peptide. While there generally no constraints on the size of the candidate nucleic acid or expression product, preferably, cyclic peptides range from about 3 to 50 amino acids in length, with from 4 to 25 being more preferred and from 5 to 10 being particularly preferred. However, in some embodiments larger cyclic peptides are preferred. In this embodiment peptides up to 200 amino acids are preferred with from 20 to 100 more preferred and from 30 to 70 amino acids particularly preferred.

In a preferred embodiment, the candidate bioactive agents are linked to a fusion partner. By “fusion partner” or “functional group” herein is meant a sequence that is associated with the candidate bioactive agent, that confers upon all members of the library in that class a common function or ability. Fusion partners can be heterologous (i.e. not native to the host cell), or synthetic (not native to any cell). Suitable fusion partners include, but are not limited to: a) presentation structures, as defined below, which provide the candidate bioactive agents in a conformationally restricted or stable form; b) targeting sequences, defined below, which allow the localization of the candidate bioactive agent into a subcellular or extracellular compartment; c) rescue sequences as defined below, which allow the purification or isolation of either the candidate bioactive agents or the nucleic acids encoding them; d) stability sequences, which confer stability or protection from degradation to the candidate bioactive agent or the nucleic acid encoding it, for example resistance to proteolytic degradation; e) dimerization sequences, to allow for peptide dimerization; f) fusion proteins such as reporter, detection or selection genes and/or proteins; or g) any combination of a), b), c), d), e), and f), as well as linker sequences as needed.

In a preferred embodiment, the fusion partner is a presentation structure. By “presentation structure” or grammatical equivalents herein is meant a sequence, which, when fused to candidate bioactive agents, causes the candidate agents to assume a conformationally restricted form. Proteins interact with each other largely through conformationally constrained domains. Although small peptides with freely rotating amino and carboxyl termini can have potent functions as is known in the art, the conversion of such peptide structures into pharmacologic agents is difficult due to the inability to predict side-chain positions for peptidomimetic synthesis. Therefore the presentation of peptides in conformationally constrained structures will benefit both the later generation of pharmaceuticals and will also likely lead to higher affinity interactions of the peptide with the target protein. This fact has been recognized in the combinatorial library generation systems using biologically generated short peptides in bacterial phage systems. A number of workers have constructed small domain molecules in which one might present randomized peptide structures.

While the candidate bioactive agents may be either nucleic acid or peptides, presentation structures are preferably used with peptide candidate agents. Thus, synthetic presentation structures, i.e. artificial polypeptides, are capable of presenting a randomized peptide as a conformationally-restricted domain. Generally such presentation structures comprise a first portion joined to the N-terminal end of the randomized peptide, and a second portion joined to the C-terminal end of the peptide; that is, the peptide is inserted into the presentation structure, although variations may be made, as outlined below. To increase the functional isolation of the randomized expression product, the presentation structures are selected or designed to have minimal biologically activity when expressed in the target cell.

Preferred presentation structures maximize accessibility to the peptide by presenting it on an exterior loop. Accordingly, suitable presentation structures include, but are not limited to, minibody structures, loops on beta-sheet turns and coiled-coil stem structures in which residues not critical to structure are randomized, zinc-finger domains, cysteine-linked (disulfide) structures, transglutaminase linked structures, cyclic peptides, B-loop structures, helical barrels or bundles, leucine zipper motifs, etc.

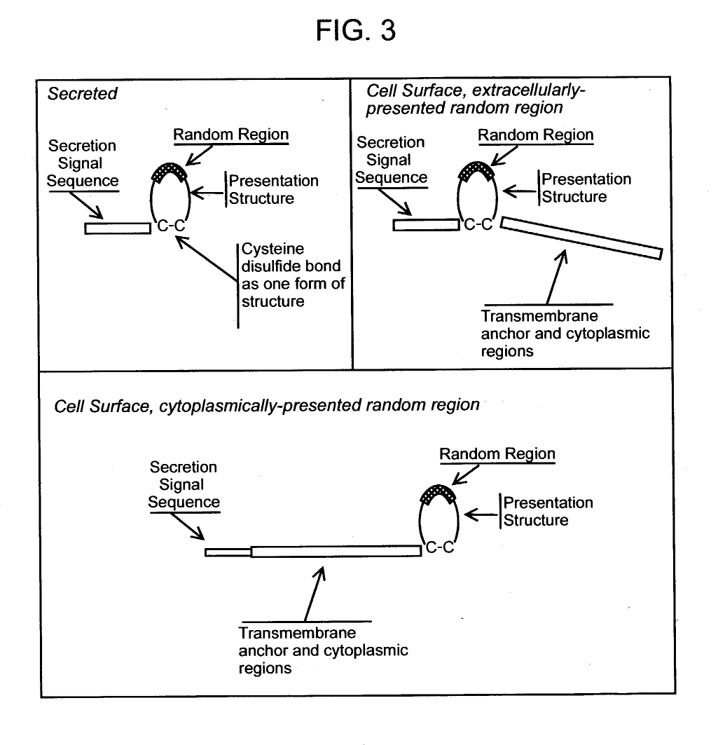

In a preferred embodiment, the presentation structure is a coiled-coil structure, allowing the presentation of the randomized peptide on an exterior loop. See, for example, Myszka et al., Biochem. 33:2362-2373 (1994), hereby incorporated by reference, and FIG. 3). Using this system investigators have isolated peptides capable of high affinity interaction with the appropriate target. In general, coiled-coil structures allow for between 6 to 20 randomized positions.

A preferred coiled-coil presentation structure is as follows: MGCAALESEVSALESEVASLESEVAALGRGDMPLAAVKSKLSAVKSKLASVKSKLAACGPP. The underlined regions represent a coiled-coil leucine zipper region defined previously (see Martin et al., EMBO J. 13(22):5303-5309 (1994), incorporated by reference). The bolded GRGDMP region represents the loop structure and when appropriately replaced with randomized peptides (i.e. candidate bioactive agents, generally depicted herein as (X)ₙ, where X is an amino acid residue and n is an integer of at least 5 or 6) can be of variable length. The replacement of the bolded region is facilitated by encoding restriction endonuclease sites in the underlined regions, which allows the direct incorporation of randomized oligonucleotides at these positions. For example, a preferred embodiment generates a XhoI site at the double underlined LE site and a HindIII site at the double-underlined KL site.

In a preferred embodiment, the presentation structure is a minibody structure. A “minibody” is essentially composed of a minimal antibody complementarity region. The minibody presentation structure generally provides two randomizing regions that in the folded protein are presented along a single face of the tertiary structure. See for example Bianchi et al., J. Mol. Biol. 236(2):649-59 (1994), and references cited therein, all of which are incorporated by reference). Investigators have shown this minimal domain is stable in solution and have used phage selection systems in combinatorial libraries to select minibodies with peptide regions exhibiting high affinity, Kd=10⁻⁷, for the pro-inflammatory cytokine IL-6.

A preferred minibody presentation structure is as follows: MGRNSQATSGFTFSHFYM EWVRGGEYIAASRHKHNKYTTEYSASVKGRYIVSRDTSQSILYLQKKKG PP. The bold, underline regions are the regions which may be randomized. The italized phenylalanine must be invariant in the first randomizing region. The entire peptide is cloned in a three-oligonucleotide variation of the coiled-coil embodiment, thus allowing two different randomizing regions to be incorporated simultaneously. This embodiment utilizes non-palindromic BstXI sites on the termini.

In a preferred embodiment, the presentation structure is a sequence that contains generally two cysteine residues, such that a disulfide bond may be formed, resulting in a conformationally constrained sequence. This embodiment is particularly preferred when secretory targeting sequences are used. As will be appreciated by those in the art, any number of random sequences, with or without spacer or linking sequences, may be flanked with cysteine residues. In other embodiments, effective presentation structures may be generated by the random regions themselves. For example, the random regions may be “doped” with cysteine residues which, under the appropriate redox conditions, may result in highly crosslinked structured conformations, similar to a presentation structure. Similarly, the randomization regions may be controlled to contain a certain number of residues to confer β-sheet or α-helical structures.

In a preferred embodiment, the fusion partner is a targeting sequence. As will be appreciated by those in the art, the localization of proteins within a cell is a simple method for increasing effective concentration and determining function. For example, RAF1 when localized to the mitochondrial membrane can inhibit the anti-apoptotic effect of BCL-2. Similarly, membrane bound Sos induces Ras mediated signaling in T-lymphocytes. These mechanisms are thought to rely on the principle of limiting the search space for ligands, that is to say, the localization of a protein to the plasma membrane limits the search for its ligand to that limited dimensional space near the membrane as opposed to the three dimensional space of the cytoplasm. Alternatively, the concentration of a protein can also be simply increased by nature of the localization. Shuttling the proteins into the nucleus confines them to a smaller space thereby increasing concentration. Finally, the ligand or target may simply be localized to a specific compartment, and inhibitors must be localized appropriately.

Thus, suitable targeting sequences include, but are not limited to, binding sequences capable of causing binding of the expression product to a predetermined molecule or class of molecules while retaining bioactivity of the expression product, (for example by using enzyme inhibitor or substrate sequences to target a class of relevant enzymes); sequences signaling selective degradation, of itself or co-bound proteins; and signal sequences capable of constitutively localizing the candidate expression products to a predetermined cellular locale, including a) subcellular locations such as the Golgi, endoplasmic reticulum, nucleus, nucleoli, nuclear membrane, mitochondria, chloroplast, secretory vesicles, lysosome, and cellular membrane; and b) extracellular locations via a secretory signal. Particularly preferred is localization to either subcellular locations or to the outside of the cell via secretion.

In a preferred embodiment, the targeting sequence is a nuclear localization signal (NLS). NLSs are generally short, positively charged (basic) domains that serve to direct the entire protein in which they occur to the cell's nucleus. Numerous NLS amino acid sequences have been reported including single basic NLS's such as that of the SV40 (monkey virus) large T Antigen (Pro Lys Lys Lys Arg Lys Val), Kalderon (1984), et al., Cell, 39:499-509; the human retinoic acid receptor-β nuclear localization signal (ARRRRP); NFKB p50 (EEVQRKRQKL; Ghosh et al., Cell 62:1019 (1990); NFKB p65 (EEKRKRTYE; Nolan et al., Cell 64:961 (1991); and others (see for example Boulikas, J. Cell. Biochem. 55(1):32-58 (1994), hereby incorporated by reference) and double basic NLS's exemplified by that of the Xenopus (African clawed toad) protein, nucleoplasmin (Ala Val Lys Arg Pro Ala Ala Thr Lys Lys Ala Gly Gln Ala Lys Lys Lys Lys Leu Asp), Dingwall, et al., Cell, 30:449-458, 1982 and Dingwall, et al., J. Cell Biol., 107:641-849; 1988). Numerous localization studies have demonstrated that NLSs incorporated in synthetic peptides or grafted onto reporter proteins not normally targeted to the cell nucleus cause these peptides and reporter proteins to be concentrated in the nucleus. See, for example, Dingwall, and Laskey, Ann, Rev. Cell Biol., 2:367-390, 1986; Bonnerot, et al., Proc. Natl. Acad. Sci. USA, 84:6795-6799, 1987; Galileo, et al., Proc. Natl. Acad. Sci. USA, 87:458-462, 1990.

In a preferred embodiment, the targeting sequence is a membrane anchoring signal sequence. This is particularly useful since many parasites and pathogens bind to the membrane, in addition to the fact that many intracellular events originate at the plasma membrane. Thus, membrane-bound peptide libraries are useful for both the identification of important elements in these processes as well as for the discovery of effective inhibitors. The invention provides methods for presenting the randomized expression product extracellularly or in the cytoplasmic space; see FIG. 3. For extracellular presentation, a membrane anchoring region is provided at the carboxyl terminus of the peptide presentation structure. The randomized epression product region is expressed on the cell surface and presented to the extracellular space, such that it can bind to other surface molecules (affecting their function) or molecules present in the extracellular medium. The binding of such molecules could confer function on the cells expressing a peptide that binds the molecule. The cytoplasmic region could be neutral or could contain a domain that, when the extracellular randomized expression product region is bound, confers a function on the cells (activation of a kinase, phosphatase, binding of other cellular components to effect function). Similarly, the randomized expression product-containing region could be contained within a cytoplasmic region, and the transmembrane region and extracellular region remain constant or have a defined function.

Membrane-anchoring sequences are well known in the art and are based on the genetic geometry of mammalian transmembrane molecules. Peptides are inserted into the membrane based on a signal sequence (designated herein as ssTM) and require a hydrophobic transmembrane domain (herein TM). The transmembrane proteins are inserted into the membrane such that the regions encoded 5′ of the transmembrane domain are extracellular and the sequences 3′ become intracellular. Of course, if these transmembrane domains are placed 5′ of the variable region, they will serve to anchor it as an intracellular domain, which may be desirable in some embodiments. ssTMs and TMs are known for a wide variety of membrane bound proteins, and these sequences may be used accordingly, either as pairs from a particular protein or with each component being taken from a different protein, or alternatively, the sequences may be synthetic, and derived entirely from consensus as artificial delivery domains.

As will be appreciated by those in the art, membrane-anchoring sequences, including both ssTM and TM, are known for a wide variety of proteins and any of these may be used. Particularly preferred membrane-anchoring sequences include, but are not limited to, those derived from CD8, ICAM-2, IL-8R, CD4 and LFA-1.

Useful sequences include sequences from: 1) class I integral membrane proteins such as IL-2 receptor beta-chain (residues 1-26 are the signal sequence, 241-265 are the transmembrane residues; see Hatakeyama et al., Science 244:551 (1989) and von Heijne et al, Eur. J. Biochem. 174:671 (1988)) and insulin receptor beta chain (residues 1-27 are the signal, 957-959 are the 30 transmembrane domain and 960-1382 are the cytoplasmic domain; see Hatakeyama, supra, and Ebina et al., Cell 40:747 (1985)); 2) class 11 integral membrane proteins such as neutral endopeptidase (residues 29-51 are the transmembrane domain, 2-28 are the cytoplasmic domain; see Malfroy et al., Biochem. Biophys. Res. Commun. 144:59 (1987)); 3) type III proteins such as human cytochrome P450 NF25 (Hatakeyama, supra); and 4) type IV proteins such as human P-glycoprotein (Hatakeyama, supra). Particularly preferred are CD8 and ICAM-2. For example, the signal sequences from CD8 and ICAM-2 lie at the extreme 5′ end of the transcript. These consist of the amino acids 1-32 in the case of CD8 (MASPLTRFLSLNLLLLGESILGSGEAKPQAP; Nakauchi et al., PNAS USA 82:5126 (1985) and 1-21 in the case of ICAM-2 (MSSFGYRTLTVALFTLICCPG; Staunton et al., Nature (London) 339:61 (1989)). These leader sequences deliver the construct to the membrane while the hydrophobic transmembrane domains, placed 3′ of the random candidate region, serve to anchor the construct in the membrane. These transmembrane domains are encompassed by amino acids 145-195 from CD8 (PQRPEDCRPRGSVKGTGLDFACDIYIWAPLAGICVALLLSLIITLICYHSR; Nakauchi, supra) and 224-256 from ICAM-2 (MVIIVTVVSVLLSLFVTSVLLCFIFGQHLRQQR; Staunton, supra).

Alternatively, membrane anchoring sequences include the GPI anchor, which results in a covalent bond between the molecule and the lipid bilayer via a glycosyl-phosphatidylinositol bond for example in DAF (PNKGSGTTSGTTRLLSGHTCFTLTGLLGTLVTMGLLT, with the bolded serine the site of the anchor; see Homans et al., Nature 333(6170):269-72 (1988), and Moran et al., J. Biol. Chem. 266:1250 (1991)). In order to do this, the GPI sequence from Thy-1 can be cassetted 3′ of the variable region in place of a transmembrane sequence.

Similarly, myristylation sequences can serve as membrane anchoring sequences. It is known that the myristylation of c-src recruits it to the plasma membrane. This is a simple and effective method of membrane localization, given that the first 14 amino acids of the protein are solely responsible for this function: MGSSKSKPKDPSQR (see Cross et al., Mol. Cell. Biol. 4(9):1834 (1984); Spencer et al., Science 262:1019-1024 (1993), both of which are hereby incorporated by reference). This motif has already been shown to be effective in the localization of reporter genes and can be used to anchor the zeta chain of the TCR. This motif is placed 5′ of the variable region in order to localize the construct to the plasma membrane. Other modifications such as palmitoylation can be used to anchor constructs in the plasma membrane; for example, palmitoylation sequences from the G protein-coupled receptor kinase GRK6 sequence (LLQRLFSRQDCCGNCSDSEEELPTRL, with the bold cysteines being palmitolyated; Stoffel et al., J. Biol. Chem 269:27791 (1994)); from rhodopsin (KQFRNCMLTSLCCGKNPLGD; Barnstable et al., J. Mol. Neurosci. 5(3):207 (1994)); and the p21H-ras 1 protein (LNPPDESGPGCMSCKCVLS; Capon et al., Nature 302:33 (1983)).

In a preferred embodiment, the targeting sequence is a lysozomal targeting sequence, including, for example, a lysosomal degradation sequence such as Lamp-2 (KFERQ; Dice, Ann. N.Y. Acad. Sci. 674:58 (1992); or lysosomal membrane sequences from Lamp-1 (MLIPIAGFFALAGLVLIVLIAYLIGRKRSHAGYQTI, Uthayakumar et al., Cell. Mol. Biol. Res. 41:405 (1995)) or Lamp-2 (LVPIAVGAALAGVLILVLLAYFIGLKHHHAGYEQF, Konecki et la., Biochem. Biophys. Res. Comm. 205:1-5 (1994), both of which show the transmembrane domains in italics and the cytoplasmic targeting signal underlined).

Alternatively, the targeting sequence may be a mitrochondrial localization sequence, including mitochondrial matrix sequences (e.g. yeast alcohol dehydrogenase III; MLRTSSLFTRRVQPSLFSRNILRLQST; Schatz, Eur. J. Biochem. 165:1-6 (1987)); mitochondrial inner membrane sequences (yeast cytochrome c oxidase subunit IV; MLSLRQSIRFFKPATRTLCSSRYLL; Schatz, supra); mitochondrial intermembrane space sequences (yeast cytochrome c1; MFSMLSKRWAQRTLSKSFYSTATGAASKSGKLTQKLVTAGVAAAGITASTLLYADSLTAEAMTA; Schatz, supra) or mitochondrial outer membrane sequences (yeast 70 kD outer membrane protein; MKSFITRNKTAILATVAATGTAIGAYYYYNQLQQQQQRGKK; Schatz, supra).

The target sequences may also be endoplasmic reticulum sequences, including the sequences from calreticulin (KDEL; Pelham, Royal Society London Transactions B; 1-10 (1992)) or adenovirus E3/19K protein (LYLSRRSFIDEKKMP; Jackson et al., EMBO J. 9:3153 (1990).

Furthermore, targeting sequences also include peroxisome sequences (for example, the peroxisome matrix sequence from Luciferase; SKL; Keller et al., PNAS USA 4:3264 (1987)); farnesylation sequences (for example, P21H-ras 1; LNPPDESGPGCMSCKCVLS, with the bold cysteine farnesylated; Capon, supra); geranylgeranylation sequences (for example, protein rab-5A; LTEPTQPTRNQCCSN, with the bold cysteines geranylgeranylated; Farnsworth, PNAS USA 91:11963 (1994)); or destruction sequences (cyclin B1; RTALGDIGN; Klotzbucher et al., EMBO J. 1:3053 (1996)).

In a preferred embodiment, the targeting sequence is a secretory signal sequence capable of effecting the secretion of the candidate translation product. There are a large number of known secretory signal sequences which are placed 5′ to the variable peptide region, and are cleaved from the peptide region to effect secretion into the extracellular space. Secretory signal sequences and their transferability to unrelated proteins are well known, e.g., Silhavy, et al. (1985) Microbiol. Rev. 49, 398-418. This is particularly useful to generate a peptide capable of binding to the surface of, or affecting the physiology of, a target cell that is other than the host cell, e.g., the cell infected with the retrovirus. In a preferred approach, a fusion product is configured to contain, in series, secretion signal peptide-presentation structure-randomized expression product region-presentation structure, see FIG. 3. In this manner, target cells grown in the vicinity of cells caused to express the library of peptides, are bathed in secreted peptide. Target cells exhibiting a physiological change in response to the presence of a peptide, e.g., by the peptide binding to a surface receptor or by being internalized and binding to intracellular targets, and the secreting cells are localized by any of a variety of selection schemes and the peptide causing the effect determined. Exemplary effects include variously that of a designer cytokine (i.e., a stem cell factor capable of causing hematopoietic stem cells to divide and maintain their totipotential), a factor causing cancer cells to undergo spontaneous apoptosis, a factor that binds to the cell surface of target cells and labels them specifically, etc.

Suitable secretory sequences are known, including signals from IL-2 (MYRMQLLSCIALSLALVTNS; Villinger et al., J. Immunol. 155:3946 (1995)), growth hormone (MATGSRTSLLLAFGLLCLPWLQEGSAFPT; Roskam et al., Nucleic Acids Res. 7:30 (1979)); preproinsulin (MALWMRLLPLLALLALWGPDPAAAFVN; Bell et al., Nature 284:26 (1980)); and influenza HA protein (MKAKLLVLLYAFVAGDQI; Sekiwawa et al., PNAS 80:3563)), with cleavage between the non-underlined-underlined junction. A particularly preferred secretory signal sequence is the signal leader sequence from the secreted cytokine IL-4, which comprises the first 24 amino acids of IL-4 as follows: MGLTSQLLPPLFFLLACAGNFVHG.

In a preferred embodiment, the fusion partner is a rescue sequence. A rescue sequence is a sequence which may be used to purify or isolate either the candidate agent or the nucleic acid encoding it. Thus, for example, peptide rescue sequences include purification sequences such as the His₆ tag for use with Ni affinity columns and epitope tags for detection, immunoprecipitation or FACS (fluoroscence-activated cell sorting). Suitable epitope tags include myc (for use with the commercially available 9E10 antibody), the BSP biotinylation target sequence of the bacterial enzyme BirA, flu tags, lacZ, and GST.

Alternatively, the rescue sequence may be a unique oligonucleotide sequence which serves as a probe target site to allow the quick and easy isolation of the retroviral construct, via PCR, related techniques, or hybridization.

In a preferred embodiment, the fusion partner is a stability sequence to confer stability to the candidate bioactive agent or the nucleic acid encoding it. Thus, for example, peptides may be stabilized by the incorporation of glycines after the initiation methionine (MG or MGG0), for protection of the peptide to ubiquitination as per Varshavsky's N-End Rule, thus conferring long half-life in the cytoplasm. Similarly, two prolines at the C-terminus impart peptides that are largely resistant to carboxypeptidase action. The presence of two glycines prior to the prolines impart both flexibility and prevent structure initiating events in the di-proline to be propagated into the candidate peptide structure. Thus, preferred stability sequences are as follows: MG(X)ₙGGPP, where X is any amino acid and n is an integer of at least four.

In one embodiment, the fusion partner is a dimerization sequence. Dimerization sequences are also described in U.S. Ser. No. 09/285,912 filed Apr. 2, 1999 and WO 00/51625, both of which are incorporated herein by reference. A dimerization sequence allows the non-covalent association of one random peptide to another random peptide, with sufficient affinity to remain associated under normal physiological conditions. This effectively allows small libraries of random peptides (for example, 10⁴) to become large libraries if two peptides per cell are generated which then dimerize, to form an effective library of 10⁸ (10⁴×10⁴). It also allows the formation of longer random peptides, if needed, or more structurally complex random peptide molecules. The dimers may be homo- or heterodimers.

Dimerization sequences may be a single sequence that self-aggregates, or two sequences, each of which is generated in a different retroviral construct. That is, nucleic acids encoding both a first random peptide with dimerization sequence 1, and a second random peptide with dimerization sequence 2, such that upon introduction into a cell and expression of the nucleic acid, dimerization sequence 1 associates with dimerization sequence 2 to form a new random peptide structure.

Suitable dimerization sequences will encompass a wide variety of sequences. Any number of protein-protein interaction sites are known. In addition, dimerization sequences may also be elucidated using standard methods such as the yeast two hybrid system, traditional biochemical affinity binding studies, or even using the present methods.

The fusion partners may be placed anywhere (i.e. N-terminal, C-terminal, internal) in the structure as the biology and activity permits.

In a preferred embodiment, the fusion partner includes a linker or tethering sequence. Linker sequences between various targeting sequences (for example, membrane targeting sequences) and the other components of the constructs (such as the randomized candidate agents) may be desirable to allow the candidate agents to interact with potential targets unhindered. For example, when the candidate bioactive agent is a peptide, useful linkers include glycine-serine polymers (including, for example, (GS)ₙ, (GSGGS)ₙ and (GGGS)ₙ, where n is an integer of at least one), glycine-alanine polymers, alanine-serine polymers, and other flexible linkers such as the tether for the shaker potassium channel, and a large variety of other flexible linkers, as will be appreciated by those in the art. Glycine-serine polymers are preferred since both of these amino acids are relatively unstructured, and therefore may be able to serve as a neutral tether between components. Secondly, serine is hydrophilic and therefore able to solubilize what could be a globular glycine chain. Third, similar chains have been shown to be effective in joining subunits of recombinant proteins such as single chain antibodies.

In addition, the fusion partners, including presentation structures, may be modified, randomized, and/or matured to alter the presentation orientation of the randomized expression product. For example, determinants at the base of the loop may be modified to slightly modify the internal loop peptide tertiary structure, which maintaining the randomized amino acid sequence.

The candidate bioactive agents as described above are encoded by candidate nucleic acids. By “candidate nucleic acids” herein is meant a nucleic acid, generally RNA when retroviral delivery vehicles are used, which can be expressed to form candidate bioactive agents; that is, the candidate nucleic acids encode the candidate bioactive agents and the fusion partners, if present. In addition, the candidate nucleic acids will also generally contain enough extra sequence to effect translation or transcription, as necessary. For a peptide library, the candidate nucleic acid generally contains cloning sites which are placed to allow in frame expression of the randomized peptides, and any fusion partners, if present, such as presentation structures. For example, when presentation structures are used, the presentation structure will generally contain the initating ATG, as a part of the parent vector. For a RNA library, the candidate nucleic acids are generally constructed with an internal CMV promoter, tRNA promoter or cell specific promoter designed for immediate and appropriate expression of the RNA structure at the initiation site of RNA synthesis. The RNA is expressed anti-sense to the direction of retroviral synthesis and is terminated as known, for example with an orientation specific terminator sequence. Interference from upstream transcription is alleviated in the target cell with the self-inactivation deletion, a common feature of certain retroviral expression systems.

Generally, the candidate nucleic acids are expressed within the cells to produce expression products of the candidate nucleic acids. As outlined above, the expression products include translation products, i.e. peptides, or transcription products, i.e. nucleic acid.

The candidate bioactive agents and candidate nucleic acids are randomized, either fully randomized or they are biased in their randomization, e.g. in nucleotide/residue frequency generally or per position. By “randomized” or grammatical equivalents herein is meant that each nucleic acid and peptide consists of essentially random nucleotides and amino acids, respectively. As is more fully described below, the candidate nucleic acids which give rise to the candidate expression products are chemically synthesized, and thus may incorporate any nucleotide at any position. Thus, when the candidate nucleic acids are expressed to form peptides, any amino acid residue may be incorporated at any position. The synthetic process can be designed to generate randomized nucleic acids, to allow the formation of all or most of the possible combinations over the length of the nucleic acid, thus forming a library of randomized candidate nucleic acids.

The library should provide a sufficiently structurally diverse population of randomized expression products to effect a probabilistically sufficient range of cellular responses to provide one or more cells exhibiting a desired response, such as reduced viral production. Accordingly, an interaction library must be large enough so that at least one of its members will have a structure that gives it affinity for some molecule, protein, or other factor whose activity is necessary for completion of the signaling pathway. Although it is difficult to gauge the required absolute size of an interaction library, nature provides a hint with the immune response: a diversity of 10⁷-10⁸ different antibodies provides at least one combination with sufficient affinity to interact with most potential antigens faced by an organism. Published in vitro selection techniques have also shown that a library size of 10⁷ to 10⁸ is sufficient to find structures with affinity for the target. A library of all combinations of a peptide 7 to 20 amino acids in length, such as proposed here for expression in retroviruses, has the potential to code for 20⁷ (10⁹) to 20²⁰. Thus, with libraries of 10⁷ to 10⁹ per ml of retroviral particles the present methods allow a “working” subset of a theoretically complete interaction library for 7 amino acids, and a subset of shapes for the 20²⁰ library. Thus, in a preferred embodiment, at least 10⁸, preferably at least 10⁷, more preferably at least 10⁸ and most preferably at least 10⁹ different expression products are simultaneously analyzed in the subject methods. Preferred methods maximize library size and diversity.

It is important to understand that in any library system encoded by oligonucleotide synthesis one cannot have complete control over the codons that will eventually be incorporated into the peptide structure. This is especially true in the case of codons encoding stop signals (TAA, TGA, TAG). In a synthesis with NNN as the random region, there is a 3/64, or 4.69%, chance that the codon will be a stop codon. Thus, in a peptide of 10 residues, there is an unacceptable high likelihood that 46.7% of the peptides will prematurely terminate. For free peptide structures this is perhaps not a problem. But for larger structures, such as those envisioned here, such termination will lead to sterile peptide expression. To alleviate this, random residues are encoded as NNK, where K=T or G. This allows for encoding of all potential amino acids (changing their relative representation slightly), but importantly preventing the encoding of two stop residues TAA and TGA. Thus, libraries encoding a 10 amino acid peptide will have a 15.6% chance to terminate prematurely. For candidate nucleic acids which are not designed to result in peptide expression products, this is not necessary.

In one embodiment, the library is fully randomized, with no sequence preferences or constants at any position. In a preferred embodiment, the library is biased. That is, some positions within the sequence are either held constant, or are selected from a limited number of possibilities. For example, in a preferred embodiment, the nucleotides or amino acid residues are randomized within a defined class, for example, of hydrophobic amino acids, hydrophilic residues, sterically biased (either small or large) residues, towards the creation of cysteines, for cross-linking, prolines for SH-3 domains, serines, threonines, tyrosines or histidines for phosphorylation sites, etc., or to purines, etc.

In a preferred embodiment, the bias is towards peptides or nucleic acids that interact with known classes of molecules. For example, when the candidate bioactive agent is a peptide, it is known that much of intracellular signaling is carried out via short regions of polypeptides interacting with other polypeptides through small peptide domains. For instance, a short region from the HIV-1 envelope cytoplasmic domain has been previously shown to block the action of cellular calmodulin. Regions of the Fas cytoplasmic domain, which shows homology to the mastoparan toxin from Wasps, can be limited to a short peptide region with death-inducing apoptotic or G protein inducing functions. Magainin, a natural peptide derived from Xenopus, can have potent anti-tumour and anti-microbial activity. Short peptide fragments of a protein kinase C isozyme (βPKC), have been shown to block nuclear translocation of βPKC in Xenopus oocytes following stimulation. And, short SH-3 target peptides have been used as psuedosubstrates for specific binding to SH-3 proteins. This is of course a short list of available peptides with biological activity, as the literature is dense in this area. Thus, there is much precedent for the potential of small peptides to have activity on intracellular signaling cascades. In addition, agonists and antagonists of any number of molecules may be used as the basis of biased randomization of candidate bioactive agents as well.

Thus, a number of molecules or protein domains are suitable as starting points for the generation of biased randomized candidate bioactive agents. A large number of small molecule domains are known, that confer a common function, structure or affinity. In addition, as is appreciated in the art, areas of weak amino acid homology may have strong structural homology. A number of these molecules, domains, and/or corresponding consensus sequences, are known, including, but are not limited to, SH-2 domains, SH-3 domains, Pleckstrin, death domains, protease cleavage/recognition sites, enzyme inhibitors, enzyme substrates, Traf, etc. Similarly, there are a number of known nucleic acid binding proteins containing domains suitable for use in the invention. For example, leucine zipper consensus sequences are known.

Where the ultimate expression product is a nucleic acid, at least 10, preferably at least 12, more preferably at least 15, most preferably at least 21 nucleotide positions need to be randomized, with more preferable if the randomization is less than perfect. Similarly, at least 5, preferably at least 6, more preferably at least 7 amino acid positions need to be randomized; again, more are preferable if the randomization is less than perfect.

In a preferred embodiment, biased SH-3 domain-binding oligonucleotides/peptides are made. SH-3 domains have been shown to recognize short target motifs (SH-3 domain-binding peptides), about ten to twelve residues in a linear sequence, that can be encoded as short peptides with high affinity for the target SH-3 domain. Consensus sequences for SH-3 domain binding proteins have been proposed. Thus, in a preferred embodiment, oligos/peptides are made with the following biases

1. XXXPPXPXX, wherein X is a randomized residue.

2. (within the positions of residue positions 11 to −2):

```
                    11  10  9  8  7  5  4  3  2
Met Glyaa11aa10 aa9 aa8 aa7 Arg Pro Leu Pro Pro
    1 0  −1  −2
hyd Pro hyd hyd Gly Gly Pro Pro STOP
atg ggc nnk nnk nnk nnk nnk aga cct ctg cct cca
sbk ggg sbk sbk gga ggc cca cct TAA1.
```

In this embodiment, the N-terminus flanking region is suggested to have the greatest effects on binding affinity and is therefore entirely randomized. “Hyd” indicates a bias toward a hydrophobic residue, i.e.—Val, Ala, Gly, Leu, Pro, Arg. To encode a hydrophobically biased residue, “sbk” codon biased structure is used. Examination of the codons within the genetic code will ensure this encodes generally hydrophobic residues. s=g,c; b=t, g, c; v=a, g, c; m=a, c; k=t, g; n=a, t, g, c.

The candidate nucleic acids are introduced into the cells to screen for bioactive agents capable of altering the phenotype of a cell associated with viral invection. By “introduced into” or grammatical equivalents herein is meant that the nucleic acids enter the cells in a manner suitable for subsequent expression of the nucleic acid. The method of introduction is largely dictated by the targeted cell type, discussed below. Exemplary methods include CaPO₄ precipitation, liposome fusion, lipofectin®), electroporation, viral infection, such as retrovirus, adenovirus, adeno-associated virus or lentivirus, etc. The candidate nucleic acids may stably integrate into the genome of the host cell (for example, with retroviral introduction, outlined below), or may exist either transiently or stably in the cytoplasm (i.e. through the use of traditional plasmids, utilizing standard regulatory sequences, selection markers, etc.). As many pharmaceutically important screens require human or model mammalian cell targets, retroviral vectors capable of transfecting such targets are preferred.

In a preferred embodiment, the candidate nucleic acids are part of a retroviral particle which infects the cells. Generally, infection of the cells is straightforward with the application of the infection-enhancing reagent polybrene, which is a polycation that facilitates viral binding to the target cell. Infection can be optimized such that each cell generally expresses a single construct, using the ratio of virus particles to number of cells. Infection follows a Poisson distribution.

In a preferred embodiment, the candidate nucleic acids are introduced into the cells using retroviral vectors. Currently, the most efficient gene transfer methodologies harness the capacity of engineered viruses, such as retroviruses, to bypass natural cellular barriers to exogenous nucleic acid uptake. The use of recombinant retroviruses was pioneered by Richard Mulligan and David Baltimore with the Psi-2 lines and analogous retrovirus packaging systems, based on NIH 3T3 cells (see Mann et al., Cell 33:153-159 (1993), hereby incorporated by reference). Such helper-defective packaging lines are capable of producing all the necessary trans proteins -gag, pol, and env- that are required for packaging, processing, reverse transcription, and integration of recombinant genomes. Those RNA molecules that have in cis the Ψ packaging signal are packaged into maturing virions. Retroviruses are preferred for a number of reasons. First, their derivation is easy. Second, unlike Adenovirus-mediated gene delivery, expression from retroviruses is long-term (adenoviruses do not integrate). Adeno-associated viruses have limited space for genes and regulatory units and there is some controversy as to their ability to integrate. Retroviruses therefore offer the best current compromise in terms of long-term expression, genomic flexibility, and stable integration, among other features. The main advantage of retroviruses is that their integration into the host genome allows for their stable transmission through cell division. This ensures that in cell types which undergo multiple independent maturation steps, such as hematopoietic cell progression, the retrovirus construct will remain resident and continue to express.

A particularly well suited retroviral transfection system is described in Mann et al., supra: Pear et al., PNAS USA 90(18):8392-6 (1993); Kitamura et al., PNAS USA 92:9146-9150 (1995); Kinsella et al., Human Gene Therapy 7:1405-1413; Hofmann et al., PNAS USA 93:5185-5190; Choate et al., Human Gene Therapy 7:2247 (1996); and WO 94/19478; and references cited therein, all of which are incorporated by reference.

In one embodiment of the invention, the library is generated in a retrovirus DNA construct backbone, as is generally described in the examples. Standard oligonucleotide synthesis is done to generate the random portion of the candidate bioactive agent, using techniques well known in the art (see Eckstein, Oligonucleotides and Analogues, A Practical Approach, IRL Press at Oxford University Press, 1991); libraries may be commercially purchased. Libraries with up to 10⁹ unique sequences can be readily generated in such DNA backbones. After generation of the DNA library, the library is cloned into a first primer. The first primer serves as a “cassette”, which is inserted into the retroviral construct. The first primer generally contains a number of elements, including for example, the required regulatory sequences (e.g. translation, transcription, promoters, etc), fusion partners, restriction endonuclease (cloning and subcloning) sites, stop codons (preferably in all three frames), regions of complementarity for second strand priming (preferably at the end of the stop codon region as minor deletions or insertions may occur in the random region), etc.

A second primer is then added, which generally consists of some or all of the complementarity region to prime the first primer and optional necessary sequences for a second unique restriction site for subcloning. DNA polymerase is added to make double-stranded oligonucleotides. The double-stranded oligonucleotides are cleaved with the appropriate subcloning restriction endonucleases and subcloned into the target retroviral vectors, described below.

Any number of suitable retroviral vectors may be used. Generally, the retroviral vectors may include: selectable marker genes under the control of internal ribosome entry sites (IRES), which allows for bicistronic operons and thus greatly facilitates the selection of cells expressing peptides at uniformly high levels; and promoters driving expression of a second gene, placed in sense or anti-sense relative to the 5′ LTR. Suitable selection genes include, but are not limited to, neomycin, blastocidin, bleomycin, puromycin, and hygromycin resistance genes, as well as self-fluorescent markers such as green fluoroscent protein, enzymatic markers such as lacZ, and surface proteins such as CD8, etc.

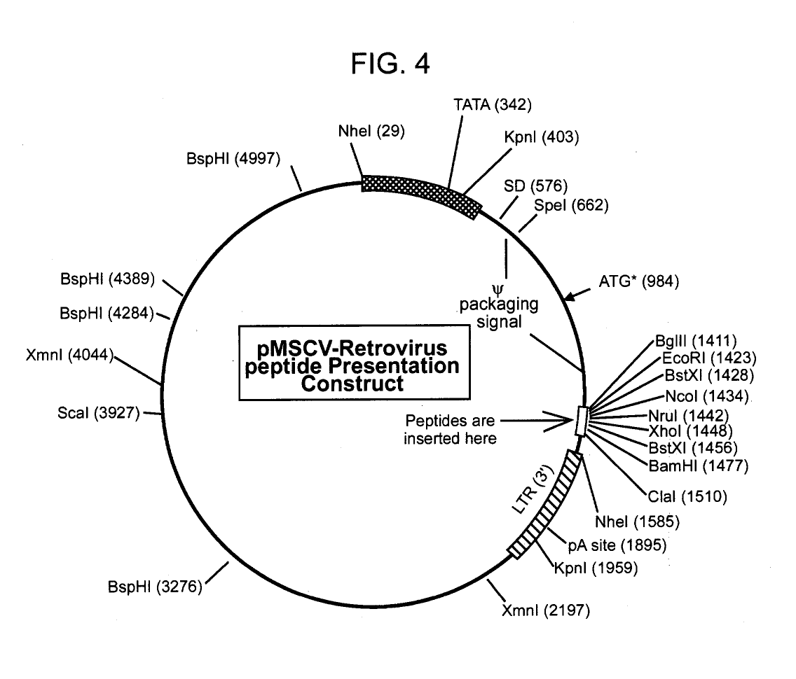

Preferred vectors include a vector based on the murine stem cell virus (MSCV) (see Hawley et al., Gene Therapy 1:136 (1994)) and a modified MFG virus (Rivere et al., Genetics 92:6733 (1995)), and pBABE, outlined in the examples. A general schematic of the retroviral construct is depicted in FIG. 4. Additional disclosure regarding vectors is found in U.S. Ser. No. 08/789,333, filed Jan. 23, 1997, which is expressly incorporated herein by reference.

The retroviruses may include inducible and constitutive promoters. For example, there are situations wherein it is necessary to induce peptide expression only during certain phases of the selection process. For instance, a scheme to provide pro-inflammatory cytokines in certain instances must include induced expression of the peptides. This is because there is some expectation that over-expressed pro-inflammatory drugs might in the long-term be detrimental to cell growth. Accordingly, constitutive expression is undesirable, and the peptide is only turned on during that phase of the selection process when the phenotype is required, and then shut the peptide down by turning off the retroviral expression to confirm the effect or ensure long-term survival of the producer cells. A large number of both inducible and constitutive promoters are known.

In addition, it is possible to configure a retroviral vector to allow inducible expression of retroviral inserts after integration of a single vector in target cells; importantly, the entire system is contained within the single retrovirus. Tet-inducible retroviruses have been diesgned incorporating the Self-Inactivating (SIN) feature of 3′ LTR enhancer/promoter retroviral deletion mutant (Hoffman et al., PNAS USA 93:5185 (1996)). Expression of this vector in cells is virtually undetectable in the presence of tetracycline or other active analogs. However, in the absence of Tet, expression is turned on to maximum within 48 hours after induction, with uniform increased expression of the whole population of cells that harbor the inducible retrovirus, indicating that expression is regulated uniformly within the infected cell population. A similar, related system uses a mutated Tet DNA-binding domain such that it bound DNA in the presence of Tet, and was removed in the absence of Tet. Either of these systems is suitable.

In this manner the primers create a library of fragments, each containing a different random nucleotide sequence that may encode a different peptide. The ligation products are then transformed into bacteria, such as E. coli, and DNA is prepared from the resulting library, as is generally outlined in Kitamura, PNAS USA 92:9146-9150 (1995), hereby expressly incorporated by reference.

Delivery of the library DNA into a retroviral packaging system results in conversion to infectious virus.

Suitable retroviral packaging system cell lines include, but are not limited to, the Bing and BOSC23 cell lines described in WO 94/19478; Soneoka et al., Nucleic Acid Res. 23(4):628 (1995); Finer et al., Blood 83:43 (1994); Pheonix packaging lines such as PhiNX-eco and PhiNX-ampho, described below; 292T+gag-pol and retrovirus envelope; PA317; and cell lines outlined in Markowitz et al., Virology 167:400 (1988), Markowitz et al., J. Virol. 62:1120 (1988), Li et al., PNAS USA 93:11658 (1996), Kinsella et al., Human Gene Therapy 7:1405 (1996), all of which are incorporated by reference.

Preferred systems include PhiNX-eco and PhiNX-ampho or similar cell lines, which are two cells lines as follows. The cell lines are based on the BING and BOSC23 cell lines described in WO 94/19478, which are based on the 293T cell line (a human embryonic kidney line transformed with adenovirus E1a and carrying a temperature sensitive T antigen co-selected with neomycin). The unique feature of this cell line is that it is highly transfectable with either calcium phosphate mediated transfection or lipid-based transfection protocols—greater than 50% of 293T cells can be transiently transfected with plasmid DNA. Thus, the cell line could be a cellular milieu in which retroviral structural proteins and genomic viral RNA could brought together rapidly for creation of helper-defective virus. 293T cells were therefore engineered with stably integrated defective constructs capable of producing gag-pol, and envelope protein for either ecotropic or amphotropic viruses. These lines were called BOSC23 and Bing, respectively. The utility of these lines was that one could produce small amounts of recombinant virus transiently for use in small-scale experimentation. The lines offered advantages over previous stable systems in that virus could be produced in days rather than months.

Two problems became apparent with these first generation lines over the two years they have been in wide use. First, gag-pol and envelope expression was unstable and the lines required vigilant checking for retroviral production capacity; second the structure of the vectors used for protein production were not considered fully “safe” for helper virus production; and third, one of the lines was shown to be inadvertently carrying a hygromycin-containing retrovirus. Although the BING and BOSC23 lines are useful in the present invention, all of these potentially problematic issues are addressed in the PhiNX second-generation lines. These lines are based on 293T cells as well, with the following improvements. First, the ability to monitor gag-pol production on a cell-by cell basis was made by introducing an IRES-CD8 surface marker expression cassette downstream of the reading frame of the gag-pol construct (other surface markers besides CD8 are also useful). IRES (internal ribosome entry site) sequences allow secondary or tertiary protein translation from a single mRNA transcript. Thus, CD8 expression is a direct reflection of intracellular gag-pol and the stability of the producer cell population's ability to produce gag-pol can be readily monitored by flow cytometry. Second, for both the gag-pol and envelope constructs non-Moloney promoters were used to minimize recombination potential with introduced retroviral constructs, and different promoters for gag-pol and envelope were used to minimize their inter-recombination potential. The promoters used were CMV and RSV. Two cell lines were created, PHEONIX-ECO and PHEONIX-AMPHO. Gag-pol was introduced with hygromycin as the co-selectable marker and the envelope proteins were introduced with diptheria resistance as the co-selectable marker. Finally, the cells were screened to find a relatively rare cell type that produced gag-pol and env in a uniform distribution, although this is not required. In addition, a line termed PHEONIX-gp has been produced that expresses only gag-pol. This line is available for further pseudotyping of retroviral virions with other envelope proteins such as gibbon ape leukemia virus envelope or Vesicular Stomatitus VSV-G protein, Xenotropic, or retargeting envelopes can also be added.

Both PHEONIX-ECO and PHEONIX-AMPHO were tested for helper virus production and established as being helper-virus free. Both lines can carry episomes for the creation of stable cell lines which can be used to produce retrovirus. Both lines are readily testable by flow cytometry for stability of gag-pol (CD8) and envelope expression; after several months of testing the lines appear stable, and do not demonstrate loss of titre as did the first-generation lines BOSC23 and Bing (partly due to the choice of promoters driving expression of gag-pol and envelope). Both lines can also be used to transiently produce virus in a few days. Thus, these new lines are fully compatible with transient, episomal stable, and library generation for retroviral gene transfer experiments. Finally, the titres produced by these lines have been tested. Using standard polybrene-enhanced retroviral infection, titres approaching or above 10⁷ per ml were observed for both PHEONIX-eco and PHEONIX-ampho when carrying episomal constructs. When transiently produced virus is made, titres are usually ½ to ⅓ that value.

These lines are helper-virus free, carry episomes for long-term stable production of retrovirus, stably produce gag-pol and env, and do not demonstrate loss of viral titre over time. In additon, PhiNX-eco and PhiNX-ampho are capable of producing titres approaching or above 1 per ml when carrying episomal constructs, which, with concentration of virus, can be enhanced to 10⁸ to 10⁹ per ml.

In a preferred embodiment, the cell lines disclosed above, and the other methods for producing retrovirus, are useful for production of virus by transient transfection. The virus can either be used directly or be used to infect another retroviral producer cell line for “expansion” of the library.

Concentration of virus may be done as follows. Generally, retroviruses are titred by applying retrovirus-containing supernatant onto indicator cells, such as NIH3T3 cells, and then measuring the percentage of cells expressing phenotypic consequences of infection. The concentration of the virus is determined by multipying the percentage of cells infected by the dilution factor involved, and taking into account the number of target cells available to obtain a relative titre. If the retrovirus contains a reporter gene, such as lacZ, then infection, integration, and expression of the recombinant virus is measured by histological staining for lacZ exprssion or by flow cytometry (FACS). In general, retroviral titres generated from even the best of the producer cells do not exceed 10⁷ per ml, unless concentration by relatively expensive or exotic apparatus. However, as it has been recently postulated that since a particle as large as a retrovirus will not move very far by brownian motion in liquid, fluid dynamics predicts that much of the virus never comes in contact with the cells to initiate the infection process. However, if cells are grown or placed on a porous filter and retrovirus is allowed to move past cells by gradual gravitometric flow, a high concentration of virus around cells can be effectively maintained at all times. Thus, up to a ten-fold higher infectivity by infecting cells on a porous membrane and allowing retrovirus supernatant to flow past them has been seen. This should allow titres of 10⁹ after concentration.

The candidate nucleic acids, as part of the retroviral construct, are introduced into the cells to screen for bioactive agents capable of altering the phenotype of a cell associated with viral infection as is described in more detail below. Accordingly, the invention also provides a cell culture wherein a culture of cells comprising the target virus or target reporter construct also contain the candidate nucleic acids. Preferably the cell culture includes both a construct that includes the IRES/reporter fusion and a second nucleic acid comprising the nucleic acid that encodes the candidate agent. In a preferred embodiment, each of the cells in the culture includes the fusion construct and each cell also includes a different candidate nucleic acid.

As will be appreciated by those in the art, the type of cells used in the present invention can vary widely. Basically, any eukaryotic cell may be used with mammalian cells being preferred, and with mouse, rat, primate and human cells being particularly preferred, although as will be appreciated by those in the art, modifications of the system by pseudotyping allows all eukaryotic cells to be used, preferably higher eukaryotes. What is important is that the cells are able to be screened for a phenotype associated with viral infection. By “phenotype associated with viral infection” is meant any phenotype that can be observed that is representative of some aspect of the virus to be screened. That is, the phenotype includes, but is not limited to cells infected with a particular virus. Alternatively, the phenotype includes assaying some reporter that is indicative of the virus. In this embodiment the reporter is fused to a viral component; a change in the reporter indicates a change in the virus.

Viruses that can be screened for agents that inhibit IRES activity include, but are not limited to picornaviruses, enterovirus such as human poliovirus 1,human coxsackievirus B3, and bovine enterovirus, rhinovorus, hepatovirus including hepatitis A virus, cardiovirus including encephalomyocarditis virus and Theiler's encephalomyelitis virus, Aphtovirus such as foot-and-mouth disease virus, equine rhinitis A virus, equine rhinitis B virus, and Parechovirus such as human echovirus 22. In addition viruses such as Cricket paralysis-like virus (insect picorna-like virus), Plautia stali intestine virus, Rhopalosiphum padi virus, cricket paralysis virus, pestivirus, bovine viral diarrhea virus, classical swine fever virus, hepacivirus, hepatitis c virus, GB virus B, Tobamovirus, Crucifer tobamovirus. In addition RNA reverse transcribing viruses are included such as retroviridae, lentivirus, simian immunodeficiency virus, human immunodeficiency virus, BLV-HTLV retroviruses, human T-lymphotropic virus type 1, mammalian type C retrovirus, Maloney murine leukemia virus, Friend murine leukemia virus, Harvey murine sarcoma virus, avian reticuloendotheliosis virus, murine leukemia virus, and Rous sarcome virus. In addition, dsDNA viruses are includes such as herpesviridae, gammaherpesvirinae, and kaposi's sarcoma-associated herpesvirus. In a preferred embodiment, hepatitis c (HCV) is the targeted virus. However, while the discussion below emphasizes the use of HCV, it should be appreciated that other viruses also can be screened by the methods described herein.

Sequences of preferred IRES's are disclosed invirology Jun. 5, 1999;258(2):249-56, Virology Jan. 10, 1995;206(1):750-4, Hum Gene Ther Nov. 1, 1997;8(16):1855-65, Virology Feb. 3, 1997;228(1):63-73, Virology Dec. 20, 1999;265(2):206-17, Mol Cell Biol 2000 July; 20(14):4990-9, Virology May 26, 1997;232(1):32-43, J Virol 1997 January; 71(1):451-7, RNA2000 Dec; 6(12):1791-807, J Virol 2000 July; 74(14):6269-77, J Virol 1988 August; 62(8):2636-43, Mol Cell Biol 1992 August; 12(8):3636-43, Nature Mar. 19, 1992;356(6366):255-7, J Gen Virol 1999 September; 80 (Pt 9):2299-309, J Virol 2000 December; 74(24):11708-16, J Gen Virol 2001 September; 82(Pt 9):2257-69, J Virol 1990 November; 64(11):5389-95, J Virol 2000 January; 74(2):773-83, J Gen Virol 1999 September; 80 (Pt 9):2337-41, J Virol 1995 October; 69(10):6400-7, J Virol 1995 October; 69(10):6400-7, J Virol 1994 February; 68(2):1066-74, J Virol 1992 March; 66(3):1476-83, J Virol 1993 June; 67(6):3338-44, J Virol 2000 July; 74(14):6242-50, J Virol 2001 January; 75(1):181-91, FEBS Lett Sep. 2, 1996;392(3):220-4, J Virol 2001 February; 75(4):1857-63, J Virol 2001 February; 75(4):1864-9, J Virol 2001 March; 75(6):2938-45, J Virol 1995April; 69(4):2214-22, J Biol Chem Sep. 1, 1995;270(35):20376-83, J Virol 2000 January; 74(2):846-50, J Virol 1999 February; 73(2):1219-26, Nature Jul. 28, 1988;334(6180):320-5, Virology 1992 June; 188(2):685-96, Virology Mar. 15, 2000;268(2):264-71, J Virol 2000 December; 74(24):11581-8, J Biol Chem Apr. 21, 2000;275(16):11899-906, J Virol 2001 December; 75(24):12141-52, and J Virol 1992 November; 66(11):6249-56, all of which are expressly incorporated herein by reference.

Preferred viral components to which the reporter genes and/or proteins are fused include nucleic acids and proteins. Preferably nucleic acids encoding the reporter are in a construct under the control of regulatory regions from the viral genome. That is, the invention includes the use of nucleic acid constructs containing isolated viral nucleic acid translationally linked to nucleic acids encoding a reporter. Preferably the chimeric molecule includes a chimeric RNA having the coding sequence of a reporter molecule under the control of a viral regulatory nucleic acids. Regulatory regions are known in the art and include promoters, 5′ untranslated regions that allow for preferential cap-dependent translation and the like. A particularly preferred regulatory domain includes the internal ribosome entry site (IRES) of the virus, which allows preferential cap-independent translation of associated RNA.

In one embodiment developing assays to screen for agents that block or inhibit IRES element activity includes constructing a reporter construct wherein a reporter gene is inserted or fused downstream of the IRES element. Monitoring the production of the reporter serves as an indication of the activity of the IRES element. While in some embodiments the reporter construct includes only IRES fused to a reporter, in other embodiments the insert of the construct includes a reporter that is inserted into the viral genome where it is under the control of the IRES. In a preferred embodiment, when the viral genome is used, the genome has been mutated such that it is no longer replication competent. That is, the virus is unable to replicate. Standard techniques in molecular biology as are known in the art are used to make these constructs. In addition, mutations that render the virus defective for replication are known.

In an alternative embodiment developing assays to screen for agents that block IRES element activity preferably requires constructing a dicistronic mRNA characterized by the presence of two different reporter genes, wherein the translation of one gene is under IRES element control and translation of the other gene is under the control of the host-cell cap structure (m.⁷ GpppG) and cellular 5′-UTR sequence. Such a construct makes it possible to identify agents, that block IRES element activity without adversely affecting the process that cells use to initiate translation of their own mRNA.

The reporter genes can be any genes that encode products that can be conveniently and reliably detected. This list is merely illustrative and in no way limits the scope of the invention since other suitable reporter genes will be known by those ordinarily skilled in the art. Commonly used detection methods include, but are not limited to, incorporation of radioisotopes, chemiluminescence, bioluminescence, calorimetric techniques and immunological procedures. Examples of appropriate reporter genes include luciferase, chloramphenicol acetyl transferase, secreted embryonic alkaline phosphatase, .beta.-galactosidase, and dihyrodofolate reductase. Reporters are measured as an indication of the activity of the IRES. Reporter activity, therefore, is considered an indication of altered HCV production, according to the invention. That is, because the activity of the IRES correlates with HCV production, the activity of the IRES serves as an indirect measure of HCN production.

By “reporter gene” or “selection gene” or grammatical equivalents herein is meant a gene that by its presence in a cell (i.e., upon expression) allows the cell to be distinguished from a cell that does not contain the reporter gene. Reporter genes can be classified into several different types, including detection genes, survival genes, death genes, cell cycle genes, cellular biosensors, proteins producing a dominant cellular phenotype, and conditional gene products. In the present invention, expression of the protein product causes the effect distinguishing between cells expressing the reporter gene and those that do not. As is more fully outlined below, additional components, such as substrates, ligands, etc., may be additionally added to allow selection or sorting on the basis of the reporter gene.

In a preferred embodiment, the distinguishable reporter gene comprises a protein that can be used as a direct label, for example a detection gene for sorting the cells or for cell enrichment by FACS. In this embodiment, the protein product of the reporter gene itself can serve to distinguish cells that are expressing the reporter gene. In one aspect, suitable reporter genes include distinguishable wildtype and variant forms of Renilla reniformis GFP, Ptilosarcus gurneyi GFP, and Renilla muelleri GFP. In another aspect, the reporter gene comprises other fluorescent proteins, such as Aequoria Victoria GFP (Chalfie, M. et al. (1994) Science 263: 802-05), EGFP; Clontech—Genbank Accession Number U55762), blue fluorescent protein (BFP; Quantum Biotechnologies, Inc. 1801 de Maisonneuve Blvd. West, 8th Floor, Montreal (Quebec) Canada H3H 1J9; Stauber, R. H. (1998) Biotechniques 24: 462-71; Heim, R. et al. (1996) Curr. Biol. 6: 178-82), enhanced yellow fluorescent protein (EYFP; 1. Clontech Laboratories, Inc., 1020 East Meadow Circle, Palo Alto, Calif. 94303), Anemonia majano fluorescent protein (amFP486, Matz, M. V. (1999) Nat. Biotech. 17: 969-73), Zoanthus fluorescent proteins (zFP506, zFP538; Matz, supra), Discosoma fluorescent protein (dsFP483, drFP583; Matz, supra), and Clavularia fluorescent protein (cFP484; Matz, supra). Other suitable reporter genes include, among others, luciferases (for example, firefly, Kennedy, H. J. et al. (1999) J. Biol. Chem. 274: 13281-91; Renilla reniformis, Lorenz, W. W. (1996) J Biolumin. Chemilumin. 11: 31-37; Renilla muelleri, U.S. Pat. No. 6,232,107), β-galactosidase (Nolan, G. et al. (1988) Proc. Natl. Acad. Sci. USA 85: 2603-07), β-glucouronidase (Jefferson, R. A. et al. (1987) EMBO J. 6: 3901-07; Gallager, S., “GUS Protocols: Using the GUS Gene as a reporter of gene expression,” Academic Press, Inc., 1992), horseradish peroxidase, alkaline phosphatase, and SEAP (i.e., the secreted form of human placental alkaline phosphatase; Cullen, B. R. et al. (1992) Methods Enzymol. 216: 362-68).

In another preferred embodiment, the gene of interest comprises a reporter gene distinguishable from rGFP or pGFP. Expressing two distinguishable, separate reporter proteins allows targeting of individual reporter proteins to distinct cellular locations, provides increased discrimination of cells expressing the fusion nucleic acid, and affords a basis for monitoring expression of the other reporter gene.

In another embodiment, the reporter gene encodes a protein that will bind a label that can be used as the basis of the cell enrichment (sorting); that is, the reporter gene serves as an indirect label or detection gene. In a preferred embodiment, the reporter gene encodes a cell-surface protein. For example, the reporter gene may be any cell-surface protein not normally expressed on the surface of the cell, such that secondary binding agents serve to distinguish cells that contain the reporter gene from those that do not. Alternatively, albeit non-preferably, reporters comprising normally expressed cell-surface proteins could be used, and differences between cells containing the reporter construct and those without could be determined. Thus, secondary binding agents bind to the reporter protein. These secondary binding agents are preferably labeled, for example with fluors, and can be antibodies, haptens, etc. For example, fluorescently labeled antibodies to the reporter gene can be used as the label. Similarly, membrane-tethered streptavidin could serve as a reporter gene, and fluorescently-labeled biotin could be used as the label, i.e., the secondary binding agent. Alternatively, the secondary binding agents need not be labeled as long as the secondary binding agent can be used to distinguish the cells containing the construct; for example, the secondary binding agents may be used in a column, and the cells passed through, such that the expression of the reporter gene results in the cell being bound to the column, and a lack of the reporter gene (i.e. inhibition), results in the cells not being retained on the column. Other suitable reporter proteins/secondary labels include, but are not limited to, antigens and antibodies, enzymes and substrates (or inhibitors), etc.

In a preferred embodiment, the reporter gene comprises a survival gene that serves to provide a nucleic acid without which the cell cannot survive, such as drug resistance genes. In this embodiment, expressing the survival gene allows selection of cells expressing the fusion nucleic acid by identifying cells that survive, for example in presence of a selection compound. Examples of drug resistance genes include, but are not limited to, puromycin resistance (puromycin-N-acetyl-transferase) (de la Luna, S. et al. (1992) Methods Enzymol. 216: 376-85), G418 neomycin resistance gene, hygromycin resistance gene (hph), and blasticidine resistance genes (bsr, brs, and BSD; Pere-Gonzalez, et al.(1990) Gene, 86: 129-34; Izumi, M. et al. (1991) Exp. Cell Res. 197: 229-33; Itaya, M. et al. (1990) J. Biochem. 107: 799-801; Kimura, M. et al. (1994) Mol. Gen. Genet. 242: 121-29). In addition, generally applicable survival genes are the family of ATP-binding cassette transporters, including multiple drug resistance gene (MDR1) (see Kane, S. E. et. al. (1988) Mol. Cell. Biol. 8: 3316-21 and Choi, K. H. et al. (1988) Cell 53: 519-29), multi-drug resistance associated proteins (MRP) (Bera, T. K. et al. (2001) Mol. Med. 7: 509-16), and breast cancer associated protein (BCRP or MXR) (Tan, B. et al. (2000) Curr. Opin. Oncol. 12: 450-58). When expressed in cells, these selectable transporter genes can confer resistance to a variety of toxic reagents, especially anti-cancer drugs (i.e., methotrexate, colchicine, tamoxifen, mitoxanthrone, and doxorubicin). As will be appreciated by those skilled in the art, the choice of the selection/survival gene will depend on the host cell type used.

In a preferred embodiment, the reporter gene comprises a death gene that causes the cells to die when expressed. Death genes fall into two basic categories: death genes that encode death proteins requiring a death ligand to kill the cells, and death genes that encode death proteins that kill cells as a result of high expression within the cell and do not require the addition of any death ligand. Preferred are cell death mechanisms that require a two-step process: the expression of the death gene and induction of the death phenotype with a signal or ligand such that the cells may be grown expressing the death gene, and then induced to die. A number of death genes/ligand pairs are known, including, but not limited to, the Fas receptor and Fas ligand (Schneider, P. et al. (1997) J. Biol. Chem. 272: 18827-33; Gonzalez-Cuadrado, S. et al. (1997) Kidney Int. 51: 1739-46; Muruve, D. A. et al. (1997) Hum. Gene Ther. 8: 955-63); p450 and cyclophosphamide (Chen, L. et al. (1997) Cancer Res. 57: 4830-37); thymidine kinase and gangcylovir (Stone, R. (1992) Science 256: 1513); and tumor necrosis factor (TNF) receptor and TNF.

When death genes requiring ligands are used, preferred embodiments use chimeric death genes (i.e, chimeric death receptor genes). Chimeric death receptors may comprise the extracellular domain of a ligand-activated multimerizing receptor and the endogenous cytoplasmic domain of a death receptor gene, such as Fas or TNF. This avoids endogenous activation of the death gene. Thus, in one embodiment, substituting the extracellular portion of a death receptor, such as Fas, with the extracellular portion of another ligand activated multimerizing receptor provides a basis for using a completely different signal to activate cell death. Suitable ligand-activated dimerizing receptors include, but are not limited to, the CD8 receptor, erythropoeitin receptor, thrombopoietin receptor, growth hormone receptor, Fas receptor, platelet derived growth hormone receptor, epidermal growth factor receptor, leptin receptor, and various interleukin receptors (e.g., IL-1, IL-2, IL-3, IL-4, IL-5, IL-6, IL-7, IL-8, IL-9, IL-11, IL-12, IL-13, IL-15, and IL-17). When particular receptors are employed to modulate promoter activity, these receptors (e.g., IL-4 when examining IL-4 induced promoter activity) are not preferred for use as a chimeric death gene receptor.

In a preferred embodiment, the chimeric cell death receptor genes are chimeric Fas receptors. The exact combination will depend on the cell type used and the receptors normally produced by these cells. For illustration, when the cells are human cells, a non-human extracellular domain and a human cytosolic domain are preferred to prevent endogenous induction of the death gene. Thus, when human cells are used, a preferred chimeric receptor gene may comprise a murine extracellular Fas receptor domain and a human cytosolic domain, such that the endogenous human Fas ligand will not activate the murine receptor domain. Alternatively, human extracellular domains may be used when the cells do not endogenously produce the cognate ligand. For example, human EPO extracellular domain may be used when cells do not endogenously produce EPO (Kawaguchi, Y. et al. (1997) Cancer Lett. 116: 53-59; Takebayashi, H. et al. (1996) Cancer Res. 56: 4164; Rudert, F. et al. (1994) Biochem Biophys Res Commun. 204:1102-10; Takahashi, T. et al. (1996) J. Biol. Chem. 271: 17555-60). In another aspect, the extracellular domains are combinations of different extracellular domains that form functional receptors (Mares, et al. (1992) Growth Factors, 6: 93-101; Seedorf, K. et al. (1991) J Biol Chem. 266: 12424-31; Heidaran, M. A. et al. (1990) J. Biol. Chem. 265: 18741-44; Okuda, K. et al. (1997) J. Clin. Invest. 100: 1708-15; Anders, R. A. et al. (1996) J. Biol. Chem. 271: 21758-66; Krishnan, K. et al. (1996) Oncogene, 13:125-33; Ohashi, et al. (1994) Proc. Natl. Acad. Sci. USA, 91: 158-62;; and Amara, J. F. et al. (1997) Proc. Natl. Acad. Sci. USA 94:10618-23. In general, the chimeric death gene receptors have a transmembrane domain. As will be appreciated by those skilled in the art, the transmembrane domain from any of the receptors can be used, although it is preferable to use the transmembrane domain associated with the chosen cytosolic domain to preserve the interaction of the transmembrane domain with other endogenous signaling proteins (Declercq, W. et al. (1995) Cytokine 7: 701-09).

Alternatively, the death genes are “one step” death genes, which need not require a ligand and death results from high expression of the gene. These death genes kill a cell without requiring a ligand or secondary signal. In one aspect, cell death is induced by the overexpression of a number of programmed cell death (PCD) proteins known to cause cell death, including, but not limited to, caspases, bax, TRADD, FADD, SCK, MEK, etc.

In another aspect, one step death genes also include toxins that cause cell death, or impair cell survival or cell function when expressed by a cell. These toxins generally do not require addition of a ligand to produce toxicity. An example of a suitable toxin is campylobacter toxin CDT (Lara-Tejero, M. (2000) Science, 290: 354-57). Expression of CdtB subunit, which has homology to nucleases, causes cell cycle arrest and ultimately cell death. Another toxin, the diptheria toxin (and similar Pseudomonas exotoxin), functions by ADP ribosylating the ef-2 (elongation factor 2) molecule in the cell and preventing translation. Expression of the diptheria toxin A subunit induces cell death in cells expressing the toxin fragment. Other useful toxins include cholera toxin and pertussis toxin (catalytic subunit-A ADP ribosylates G proteins that regulate adenylate cyclase), pierisin from cabbage butterflys (induces apoptosis in mammalian cells; Watanabe, M. (1999) Proc. Natl. Acad. Sci. USA 96: 10608-13), phospholipase snake venom toxins (Diaz, C. et al. (2001) Arch. Biochem. Biophys. 391: 56-64), ribosome inactivating toxins (i.e. ricin A chain, Gluck, A. et al. (1992) J. Mol. Biol. 226: 411-24;and nigrin, Munoz, R. et al. (2001) Cancer Lett. 167: 163-69) and pore forming toxins (hemolysin and leukocidin). When the target cells are neuronal cells, neuronal specific toxins may be used to inhibit specific neuronal functions. These include bacterial toxins such as botulinum toxin and tetanus toxin, which are proteases that act on synaptic vesicle associated proteins (i.e., synaptobrevin) to prevent neurotransmitter release (see Binz, T. et al. (1994) J. Biol. Chem. 269: 9153-58; Lacy, D. B. et al. (1998) Curr. Opin. Struct. Biol. 8: 778-84).

Another preferred embodiment of a reporter gene is a cell cycle gene, that is, a gene that causes alterations in the cell cycle. For example, Cdk interacting protein p21 (see Harper, J. W. et al. (1993) Cell 75: 805-16), which inhibits cyclin dependent kinases, does not cause cell death but causes cell-cycle arrest. Thus, expressing p21 allows selecting for regulators of promoter activity or regulators of p21 activity based on detecting cells that grow out much more quickly due to low p21 activity, either through inhibiting promoter activity or inactivation of p21 protein activity. As will be appreciated by those in the art, it is also possible to configure the system to select cells based on their inability to grow out due to increased p21 activity. Similar mitotic inhibitors include p27, p57, p16, p15, p18 and p19, p19 ARF (or its human homolog p14 ARF). Other cell cycle proteins useful for altering cell cycle include cyclins (Cln), cyclin dependent kinases (Cdk), cell cycle checkpoint proteins (i.e., Rad17, p53), Cks1 p9, Cdc phosphatases (i.e., Cdc 25), etc.

In yet another preferred embodiment, the reporter gene encodes a cellular biosensor. In these fusion nucleic acids, at least one of the genes of interest may encode a rGFP or pGFP fusion polypeptide, which is itself a cellular biosensor, or the cellular biosensor may be expressed in addition to the rGFP or pGFP (or rGFP or pGFP fusion protein). By a “cellular biosensor” herein is meant a gene product that when expressed within a cell can provide information about a particular cellular state. Biosensor proteins allow rapid determination of changing cellular conditions, for example Ca⁺² levels in the cell, pH within cellular organelles, and membrane potentials (see Miesenbock, G. et al. (1998) Nature 394: 192-95; U.S. Pat. No. 6,150,176). An example of an intracellular biosensor is Aequorin, which emits light upon binding to Ca⁺² ions. The intensity of light emitted depends on the Ca⁺² concentration, thus allowing measurement of transient calcium concentrations within the cell. When directed to particular cellular organelles by fusion partners, as more fully described below, the light emitted by Aequorin provides information about Ca⁺² concentrations within the particular organelle. Other intracellular biosensors are chimeric GFP molecules engineered for fluorescence resonance energy transfer (FRET) upon binding of an analyte, such as Ca⁺² (U.S. Pat. No. 6,197,928; Miyawaki, A. et al. (1997) Nature 388: 882-87; Miyakawa, A. et al. (1997) Mol. Cell. Biol. 8: 2659-76). For example, cameleon comprises a blue or cyan mutant of GFP, calmodulin, CaM binding domain of myosin light chain kinase, and a green or yellow GFP. Upon binding of Ca⁺² by the CaM domain, FRET occurs between the two GFPs because of a structural change in the chimera. Thus, FRET intensity is dependent on the Ca⁺² levels within the cell or organelle (Kerr, R. et al. Neuron (2000) 26: 583-94). Other examples of intracellular biosensors include sensors for detecting changes in cell membrane potential (Siegel, M. et al. (1997) Neuron 19: 735-41; Sakai, R. (2001) Eur. J. Neurosci. 13: 2314-18), monitoring exocytosis (Miesenbrock, G. et al. (1997) Proc. Natl. Acad. Sci. USA 94: 3402-07), and measuring intracellular/organellar ATP concentrations via luciferase protein (Kennedy, H. J. et al. (1999) J. Biol. Chem. 274: 13281-91). These biosensors find use in monitoring the effects of various cellular effectors, for example pharmacological agents that modulate ion channel activity, neurotransmitter release, ion fluxes within the cell, and changes in ATP metabolism.

Other intracellular biosensors comprise detectable gene products with sequences that are responsive to changes in intracellular signals. These sequences include peptide sequences acting as substrates for protein kinases, peptides with binding regions for second messengers, and protein interaction sequences sensitive to intracellular signaling events (see for example, U.S. Pat. No. 5,958,713 and U.S. Pat. No. 5,925,558). For example, a fusion protein construct comprising a GFP and a protein kinase recognition site allows detecting intracellular protein kinase activity by measuring changes in GFP fluorescence arising from phosphorylation of the fusion construct. Alternatively, the GFP is fused to a protein interaction domain whose interaction with cellular components are altered by cellular signaling events. For example, it is well known that inositol-triphosphate (InsP3) induces release of Ca⁺² from intracellular stores into the cytoplasm, which results in activation of a kinases responsible for regulating various cellular responses. The precursor to InsP3 is phosphatidyl-inositol-4,5-bisphosphate (PtdInsP₂), which is localized in the plasma membrane and cleaved by phospholipase C (PLC) following activation of an appropriate receptor. Many signaling enzymes are sequestered in the plasma membrane through pleckstrin homology domains that bind specifically to PtdInsP₂. Following cleavage of PtdInsP₂, the signaling proteins translocate from the plasma membrane into the cytosol where they activate various cellular pathways. Thus, a reporter molecule such as GFP fused to a pleckstrin domain will act as a intracellular sensor for phospholipase C activation (see Haugh, J. M. et al. (2000) J. Cell. Biol. 15:1269-80; Jacobs, A. R. et al. (2001) J. Biol. Chem. 276: 40795-802; and Wang, D. S. et al. (1996) Biochem. Biophys. Res. Commun. 225: 420-26). Other similar constructs are useful for monitoring activation of other signaling cascades and are applicable as assays in screens for candidate agents that inhibit or activate particular signaling pathways.

Since protein interaction domains, such as the described pleckstrin homology domain, are important mediators of cellular responses and biochemical processes, other preferred genes of interest are proteins containing protein-interaction domains. By “protein-interaction domain” herein is meant a polypeptide region that interacts with other biomolecules, including other proteins, nucleic acids, lipids, etc. These protein domains frequently act to provide regions that induce formation of specific multiprotein complexes for recruiting and confining proteins to appropriate cellular locations or affect specificity of interaction with targets ligands, such as protein kinases and their substrates. Thus, many of these protein domains are found in signaling proteins. Protein-interaction domains comprise modules or micro-domains ranging about 20-150 amino acids that can be expressed in isolation and bind to their physiological partners. Many different interaction domains are known, most of which fall into classes related by sequence or ligand binding properties. Accordingly, the genes of interest comprising interaction domains may comprise proteins that are members of these classes of protein domains and their relevant binding partners. These domains include, among others, SH2 domains (src homology domain 2), SH3 domain (src homology domain 3), PTB domain (phosphotyrosine binding domain), FHA domain (forkedhead associated domain), WW domain, 14-3-3 domain, pleckstrin homology domain, C1 domain, C2 domain, FYVE domain (Fab-1, YGL023, Vps27, and EEA1), death domain, death effector domain, caspase recruitment domain, Bcl-2 homology domain, bromo domain, chromatin organization modifier domain, F box domain, hect domain, ring domain (Zn⁺² finger binding domain), PDZ domain (PSD-95, discs large, and zona occludens domain), sterile α motif domain, ankyrin domain, arm domain (armadillo repeat motif), WD 40 domain and EF-hand (calretinin), PUB domain (Suzuki T. et al. (2001) Biochem. Biophys. Res. Commun. 287: 1083-87), nucleotide binding domain, Y Box binding domain, H. G. domain, all of which are well known in the art.

Since protein interactions domains are pervasive in cellular signal transduction cascades and other cellular processes, such as cell cycle regulation and protein degradation, expression of single proteins or multiple proteins with interaction domains acting in specific signaling or regulatory pathway may provide a basis for inactivating, activating, or modulating such pathways in normal and diseased cells. In another aspect, the preferred embodiments comprise binding partners of these interactions domains, which are well known to those skilled in the art or are identifiable by well known methods (i.e. yeast two hybrid technique, co-precipitation of immune complexes, etc.).

Included within the protein-interaction domains are transcriptional activation domains capable of activating transcription when fused to an appropriate DNA binding domain. Transcriptional activation domains are well known in the art. These include activator domains from GAL4 (amino acids 1-147; Fields, S. et al. (1989) Nature 340: 245-46; Gill, G. et al. (1990) Proc. Natl. Acad. Sci. USA 87: 2127-31), GCN4 (Hope, I. A. et al. (1986) Cell 46: 885-94), ARD1 (Thukral, S. K. et al. (1989) Mol. Cell. Biol. 9: 2360-69), human estrogen receptor (Kumar, V. et al. (1987) Cell 51: 941-51), VP16 (Triezenberg, S. J. et al. (1988) Genes Dev. 2: 718-29), Sp1 (Courey, A. J. (1988) Cell 55: 887-98), AP-2 (Williams, T. et al. (1991) Genes Dev. 5: 670-82), and NF-kB p65 subunit and related Rel proteins (Moore, P. A. et al. (1993) Mol. Cell. Biol. 13: 1666-74). DNA binding domains include, among others, leucine zipper domain, homeo box domain, Zn⁺² finger domain, paired domain, LIM domain, ETS domain, and T Box domain.

Since the reporter gene may comprise DNA binding domains and transcriptional activation domains, other reporter genes useful for expression in the present invention are transcription factors. Preferred transcription factors are those producing a cellular phenotype when expressed within a particular cell type. Transcription factors as defined herein include both transcriptional activator or inhibitors. As not all cells will respond to expression of a particular transcription factor, those skilled in the art can choose appropriate cell strains in which expression of a transcription factor results in dominant or altered phenotypes as described below.

In another aspect, the transcription factor regulates expression of a different promoter of interest on an expression vector that does not encode the transcription factor. This arrangement requires introducing into a single cell a plurality or multiple vectors, as described below, one of which expresses the transcription factor regulating the different promoter of interest. Expression of the transcription factor is made inducible or the transcription factor itself is an inducible transcription factor, thus allowing further regulation of the different promoter of interest.

In an alternative embodiment, the transcription factor encoded by the reporter gene regulates the promoter on the expression vector encoding the transcription factor. Thus, these constructs are autoregulatory for expression of the fusion nucleic acid (Hofmann, A. (1996) Proc. Natl. Acad. Sci. USA 93: 5185-90). Accordingly, if the transcription factor inhibits the promoter activity on the expression vector, continued synthesis of transcription factor restricts expression of the fusion nucleic acid. On the other hand, if the transcription factor activates transcription, synthesis is elevated because of continued synthesis of the transcriptional activator. Consequently, by use of separation sequences to express a plurality of genes of interest, one of which encodes the transcription factor, the retroviral vector autoregulates expression of the genes of interest. To enhance autoregulation, the transcription factor is an inducible transcription factor, for example a tetracycline or steroid inducible transcription factor (e.g., RU-486 or ecdysone inducible, see White J H (1997) Adv. Pharmacol. 40: 339-67). Incorporation of an inducible transcription factor in a retroviral vector as a single autoregulatory cassette eliminates the need for additional vectors for regulating the promoter activity. Moreover, this system results in rapid, uniform expression of the gene(s) of interest.

In another preferred embodiment, the reporter gene encodes a protein whose expression has a dominant effect on the cell (i.e., produces an altered cellular phenotype). By “dominant effect” herein is meant that the protein or peptide produces an effect upon the cell in which it is expressed, or on another cell not expressing the dominant effect protein, and is detected by the methods described below. The dominant effect may act directly on the cell to produce the phenotype or act indirectly on a second molecule, which leads to a specific phenotype. Dominant effect is produced by introducing into cells small molecule effectors, expressing a single protein, or by expressing multiple proteins acting in combination (e.g., proteins acting synergistically on a cellular pathway or a multisubunit protein effector). As is well known in the art, expression of a variety of genes of interest may produce a dominant effect. Expressed proteins may be mutant proteins that are constitutive for a biological activity (Segouffin-Cariou, C. et al. (2000) J. Biol. Chem. 275: 3568-76; Luo et al. (1997) Mol. Cell. Biol. 17: 1562-71) or are inactive forms that sequester or inhibit activity of normal binding partners (Bossu, P. (2000) Oncogene, 19: 2147-54; Mochizuki, H. (2001) Proc. Natl. Acad. Sci. USA 98: 10918-23). The inactive forms as defined herein include expression of small modular protein-interaction regions or other domains that bind to binding partners in the cell (see for example, Gilchrist, A. et al. (1999) J. Biol. Chem. 274: 6610-16). Dominant effects are also produced by overexpression of normal cellular proteins, expression of proteins not normally expressed in a particular cell type, or expression of normally functioning proteins in cells lacking functional proteins due to mutations or deletions (Takihara, Y. et al. (2000) Carcinogenesis 21: 2073-77; Kaplan, J. B. (1994) Oncol. Res. 6: 611-15). Random peptides or biased random peptides introduced into cells can also produce dominant effects. An exemplary effect of a dominant effect by a peptide is random peptides which bind to Src SH3 domain resulting in increased Src activity. This activation is due to the peptides' antagonistic effect on negative regulation of Src (see Sparks, A. B. et al. (1994) J Biol Chem. 269: 23853-56).

As defined herein, dominant effect is not restricted to the effect on the cell expressing the protein. A dominant effect may be on a cell contacting the expressing cell or by secretion of the protein encoded by the gene of interest into the cellular medium. Proteins with dominant effect on other cells are conveniently directed to the plasma membrane or secretion by incorporating appropriate secretion and/or membrane localization signals. These membrane bound or secreted dominant effector proteins may comprise cytokines and chemokines, growth factors, toxins (e.g., neurotoxins), extracellular proteases (e.g., metalloproteases), cell surface receptor ligands (e.g., sevenless type receptor ligands), adhesion proteins (e.g., L1, cadherins, integrins, laminin), etc.

In an alternative embodiment, the reporter gene encodes a conditional gene product. By “conditional gene” product herein is meant a gene product whose activity is only apparent under certain conditions, for example at particular ranges of temperature. Other factors that conditionally affect activity of a protein include, but are not limited to, ion concentration, pH, and light (see Hager, A. (1996) Planta 198: 294-99; Pavelka J. (2001) Bioelectromagnetics 22: 371-83). A conditional gene product produces a specific cellular phenotype under a restrictive condition. In contrast, the conditional gene product does not produce a specific phenotype under permissive conditions. Methods for making or isolating conditional gene products are well known (see for example White, D. W. et al. (1993) J. Virol. 67:6876-81; Parini, M. C. (1999) Chem. Biol. 6: 679-87).

As is appreciated by those skilled in the art, conditional gene products are useful in examining genes that are detrimental to a cell's survival or in examining cellular biochemical and regulatory pathways in which the gene product functions. For those gene products that affect cell survival, use of conditional gene products allow survival of the cells under permissive conditions, but results in lethality or detriment at the restrictive condition. This feature allows screens at the restrictive condition for candidate agents, such as proteins and small molecules that may directly or indirectly suppress the effect of a conditional gene product but permit maintenance and growth of cells under permissive conditions. In addition, conditional gene products are also useful in screens for regulators of cell physiology when the conditional gene product is a participant in a cellular regulatory pathway. At the restrictive condition, the conditional gene product ceases to function or becomes activated, resulting in an altered cell phenotype due to dysregulation of the regulatory pathway. Candidate agents are then screened for their ability to activate or inhibit downstream pathways to bypass the disrupted regulatory point. Conditional gene products are well known in the art and include, among others, proteins such as dynamin involved in endocytic pathway (Damke, H. et al. (1995) Methods Enzymol. 257: 209-20), p53 involved in tumor suppression (Pochampally, R. et al. (2000) Biochem. Biophys. Res. Comm. 279: 1001-10 and Buckbinder, L. et al. (1994) Proc. Natl. Acad. Sci. USA 91: 10640-44), Vac1 involved in vesicle sorting, proteins involved in viral pathogenesis (SV40 Large T Antigen; Robinson C.C. (1980). J Virol. 35: 246-48), and gene products involved in regulating the cell cycle, such as ubiquitin conjugating enzyme CDC 34 (Ellison, K. S. et al. (1991) J. Biol. Chem. 266: 24116-20).

Preferred reporters include green fluorescent protein, particularly when it is an Aequorea GFP or a Renilla GFP, and more preferably when it is a Renilla muller GFP that is codon-optimized for expression in eukaryotic cells. Additional GFP disclosure is found in U.S. S No. 60/164,592, filed Nov. 10, 1999, Ser. No. 09/710,058, filed Nov. 10, 2000, and No. 60/290,287, May 10, 2001.

In some embodiments the HCV is assayed directly. That is, cells infected with HCV, also known as “replicons” are infected with the library of bioactive agents as described herein. Subsequently, a phenotype is screened that reflects HCV viability or HCV production.

Thus, in a preferred embodiment, replicons containing full length HCV genomes are screened. Replicons are described in detail in Lohmann et al. (1999) Science 285:110-113; Lohmann et al. (2001) J. Virol. 75: 1437-1449, both of which are expressly incorporated herein by reference.

As is more fully described below, a screen will be set up such that the cells exhibit a detectable phenotype indicative of in the presence of a bioactive agent. As is more fully described below, cell types implicated in a wide variety of disease conditions are particularly useful, so long as a suitable screen may be designed to allow the selection of cells that exhibit an altered phenotype as a consequence of the presence of a bioactive agent within the cell.

Accordingly, suitable cell types include, but are not limited to, tumor cells of all types (particularly melanoma, myeloid leukemia, carcinomas of the lung, breast, ovaries, colon, kidney, prostate, pancreas and testes), cardiomyocytes, endothelial cells, epithelial cells, lymphocytes (T-cell and B cell), mast cells, eosinophils, vascular intimal cells, hepatocytes, leukocytes including mononuclear leukocytes, stem cells such as haemopoetic, neural, skin, lung, kidney, liver and myocyte stem cells (for use in screening for differentiation and de-differentiation factors), osteoclasts, chondrocytes and other connective tissue cells, keratinocytes, melanocytes, liver cells, kidney cells, and adipocytes. Suitable cells also include known research cells, including, but not limited to, Jurkat T cells, NIH3T3 cells, CHO, Cos, etc. See the ATCC cell line catalog, hereby expressly incorporated by reference.

In one embodiment, the cells may be genetically engineered, that is, contain exogeneous nucleic acid, for example, to contain target molecules.

In a preferred embodiment, a first plurality of cells is screened. That is, the cells into which the candidate nucleic acids are introduced are screened for an altered phenotype. Thus, in this embodiment, the effect of the bioactive agent is seen in the same cells in which it is made; i.e. an autocrine effect.

By a “plurality of cells” herein is meant roughly from about 10³ cells to 10⁸ or 10⁹, with from 10⁶ to 10⁸ being preferred. This plurality of cells comprises a cellular library, wherein generally each cell within the library contains a member of the retroviral molecular library, i.e. a different candidate nucleic acid, although as will be appreciated by those in the art, some cells within the library may not contain a retrovirus, and some may contain more than one. When methods other than retroviral infection are used to introduce the candidate nucleic acids into a plurality of cells, the distribution of candidate nucleic acids within the individual cell members of the cellular library may vary widely, as it is generally difficult to control the number of nucleic acids which enter a cell during electroporation, etc.

In a preferred embodiment, the candidate nucleic acids are introduced into a first plurality of cells, and the effect of the candidate bioactive agents is screened in a second or third plurality of cells, different from the first plurality of cells, i.e. generally a different cell type. That is, the effect of the bioactive agents is due to an extracellular effect on a second cell; i.e. an endocrine or paracrine effect. This is done using standard techniques. The first plurality of cells may be grown in or on one media, and the media is allowed to touch a second plurality of cells, and the effect measured. Alternatively, there may be direct contact between the cells. Thus, “contacting” is functional contact, and includes both direct and indirect. In this embodiment, the first plurality of cells may or may not be screened.

If necessary, the cells are treated to conditions suitable for the expression of the candidate nucleic acids (for example, when inducible promoters are used), to produce the candidate expression products, either translation or transcription products.

Thus, the methods of the present invention comprise introducing a molecular library of randomized candidate nucleic acids into a plurality of cells, a cellular library. Each of the nucleic acids comprises a different, generally randomized, nucleotide sequence. The plurality of cells is then screened, as is more fully outlined below, for a cell exhibiting an altered phenotype. The altered phenotype is due to the presence of a bioactive agent.

Once the replicons are infected or otherwise exposed to the library of bioactive agents, the cells are screened for an altered phenotype associated with HCV. By “altered phenotype” or “changed physiology” or other grammatical equivalents herein is meant that the phenotype of the cell is altered in some way, preferably in some detectable and/or measurable way. As will be appreciated in the art, a strength of the present invention is the wide variety of cell types and potential phenotypic changes which may be tested using the present methods. Accordingly, any phenotypic change which may be observed, detected, or measured may be the basis of the screening methods herein. Suitable phenotypic changes include, but are not limited to: gross physical changes such as changes in cell morphology, cell growth, cell viability, adhesion to substrates or other cells, and cellular density; changes in the expression of one or more RNAs, proteins, lipids, hormones, cytokines, or other molecules; changes in the equilibrium state (i.e. half-life) or one or more RNAs, proteins, lipids, hormones, cytokines, or other molecules; changes in the localization of one or more RNAs, proteins, lipids, hormones, cytokines, or other molecules; changes in the bioactivity or specific activity of one or more RNAs, proteins, lipids, hormones, cytokines, receptors, or other molecules; changes in the secretion of ions, cytokines, hormones, growth factors, or other molecules; alterations in cellular membrane potentials, polarization, integrity or transport; changes in infectivity, susceptability, latency, adhesion, and uptake of viruses and bacterial pathogens; etc. By “capable of altering the phenotype” herein is meant that the bioactive agent can change the phenotype of the cell in some detectable and/or measurable way. Preferred phenotypes to be screened include replicon viability, viral protein production, viral mRNA production, and the like. Proteins can be detected by immunoblot analysis, synthesis of labeled proteins, i.e. with ³⁵S labeled amino acids, or other methods known to the skilled artisan. mRNA levels are measured generally by RNA blot analysis or more preferably by PCR analysis, for example by TaqMan™ analysis. Reduced mRNA production or protein synthesis provides an indication that the bioactive agent has interfered with or inhibited translation of the viral RNA. This in turn suggests that the IRES has been inhibited.

The altered phenotype may be detected in a wide variety of ways, as is described more fully below, and will generally depend and correspond to the phenotype that is being changed. Generally, the changed phenotype is detected using, for example: microscopic analysis of cell morphology; standard cell viability assays, including both increased cell death and increased cell viability, for example, cells that are now resistant to cell death via virus, bacteria, or bacterial or synthetic toxins; standard labeling assays such as fluorometric indicator assays for the presence or level of a particular cell or molecule, including FACS or other dye staining techniques; biochemical detection of the expression of target compounds after killing the cells; etc. In some cases, as is more fully described herein, the altered phenotype is detected in the cell in which the randomized nucleic acid was introduced; in other embodiments, the altered phenotype is detected in a second cell which is responding to some molecular signal from the first cell.

In preferred embodiments of this aspect of the invention, methods of screening for bioactive agents capable of modulating the following physiological processes or biochemical activities are provided: IgE production in B cells; mast cell activation by IgE binding; mast cell degranulation; B cell activation and antibody secretion in response to antigen receptor stimulation; T cell activation in response to antigen receptor stimulation; epithelial cell activation; E3 ubiquitin ligase activity; inflammation induced by E3 ubiquitin ligase activity; inflammation induced by TNF activity; apoptosis in activated T cells; angiogenesis; uncontrolled cell proliferation; uncontrolled cell proliferation mediated by E3 ubiquitin ligase activity; and translation of Hepatitis C-encoded proteins.

In a preferred embodiment, once a cell with an altered phenotype is detected, the cell is isolated from the plurality which do not have altered phenotypes. This may be done in any number of ways, as is known in the art, and will in some instances depend on the assay or screen. Suitable isolation techniques include, but are not limited to, FACS, lysis selection using complement, cell cloning, scanning by Fluorimager, expression of a “survival” protein, induced expression of a cell surface protein or other molecule that can be rendered fluorescent or taggable for physical isolation; expression of an enzyme that changes a non-fluorescent molecule to a fluorescent one; overgrowth against a background of no or slow growth; death of cells and isolation of DNA or other cell vitality indicator dyes, etc.

In a preferred embodiment, the candidate nucleic acid and/or the bioactive agent is isolated from the positive cell. This may be done in a number of ways. In a preferred embodiment, primers complementary to DNA regions common to the retroviral constructs, or to specific components of the library such as a rescue sequence, defined above, are used to “rescue” the unique random sequence. Alternatively, the bioactive agent is isolated using a rescue sequence. Thus, for example, rescue sequences comprising epitope tags or purification sequences may be used to pull out the bioactive agent, using immunoprecipitation or affinity columns. In some instances, as is outlined below, this may also pull out the primary target molecule, if there is a sufficiently strong binding interaction between the bioactive agent and the target molecule. Alternatively, the peptide may be detected using mass spectroscopy.

Once rescued, the sequence of the bioactive agent and/or bioactive nucleic acid is determined. This information can then be used in a number of ways.

In a preferred embodiment, the bioactive agent is resynthesized and reintroduced into the target cells, to verify the effect. This may be done using retroviruses, or alternatively using fusions to the HIV-1 Tat protein, and analogs and related proteins, which allows very high uptake into target cells. See for example, Fawell et al., PNAS USA 91:664 (1994); Frankel et al., Cell 55:1189 (1988); Savion et al., J. Biol. Chem. 256:1149 (1981); Derossi et al., J. Biol. Chem. 269:10444 (1994); and Baldin et al., EMBO J. 9:1511 (1990), all of which are incorporated by reference.

In a preferred embodiment, the sequence of a bioactive agent is used to generate more candidate bioactive agents. For example, the sequence of the bioactive agent may be the basis of a second round of (biased) randomization, to develop bioactive agents with increased or altered activities. Alternatively, the second round of randomization may change the affinity of the bioactive agent. Furthermore, it may be desirable to put the identified random region of the bioactive agent into other presentation structures, or to alter the sequence of the constant region of the presentation structure, to alter the conformation/shape of the bioactive agent. It may also be desirable to “walk” around a potential binding site, in a manner similar to the mutagenesis of a binding pocket, by keeping one end of the ligand region constant and randomizing the other end to shift the binding of the peptide around.

In a preferred embodiment, either the bioactive agent or the bioactive nucleic acid encoding it is used to identify target molecules, i.e. the molecules with which the bioactive agent interacts. As will be appreciated by those in the art, there may be primary target molecules, to which the bioactive agent binds or acts upon directly, and there may be secondary target molecules, which are part of the signaling pathway affected by the bioactive agent; these might be termed “validated targets”. In addition the targets may be host cell proteins or nucleic acids. Targets may also include viral components such as viral nucleic acids or proteins. When host cell machinery is implicated, it is thought that alteration of the host cell molecules affects IRES regulated translation of the viral proteins.

In a preferred embodiment, the bioactive agent is used to pull out target molecules. For example, as outlined herein, if the target molecules are proteins, the use of epitope tags or purification sequences can allow the purification of primary target molecules via biochemical means (co-immunoprecipitation, affinity columns, etc.). Alternatively, the peptide, when expressed in bacteria and purified, can be used as a probe against a bacterial cDNA expression library made from mRNA of the target cell type. Or, peptides can be used as “bait” in either yeast or mammalian two or three hybrid systems. Such interaction cloning approaches have been very useful to isolate DNA-binding proteins and other interacting protein components. The peptide(s) can be combined with other pharmacologic activators to study the epistatic relationships of signal transduction pathways in question. It is also possible to synthetically prepare labeled peptide bioactive agent and use it to screen a cDNA library expressed in bacteriophage for those cDNAs which bind the peptide. Furthermore, it is also possible that one could use cDNA cloning via retroviral libraries to “complement” the effect induced by the peptide. In such a strategy, the peptide would be required to be stochiometrically titrating away some important factor for a specific signaling pathway. If this molecule or activity is replenished by over-expression of a cDNA from within a cDNA library, then one can clone the target. Similarly, cDNAs cloned by any of the above yeast or bacteriophage systems can be reintroduced to mammalian cells in this manner to confirm that they act to complement function in the system the peptide acts upon.

Once primary target molecules have been identified, secondary target molecules may be identified in the same manner, using the primary target as the “bait”. In this manner, signaling pathways may be elucidated. Similarly, bioactive agents specific for secondary target molecules may also be discovered, to allow a number of bioactive agents to act on a single pathway, for example for combination therapies.

The screening methods of the present invention may be useful to screen a large number of cell types under a wide variety of conditions. Generally, the host cells are cells that are involved in disease states, and they are tested or screened under conditions that normally result in undesirable consequences on the cells. When a suitable bioactive agent is found, the undesirable effect may be reduced or eliminated. Alternatively, normally desirable consequences may be reduced or eliminated, with an eye towards elucidating the cellular mechanisms associated with the disease state or signaling pathway.

In preferred embodiments, methods of screening for bioactive agents capable of modulating the following physiological processes or biochemical activities are provided: IgE production in B cells; mast cell activation by IgE binding; mast cell degranulation; B cell activation and antibody secretion in response to antigen receptor stimulation; T cell activation in response to antigen receptor stimulation; epithelial cell activation; E3 ubiquitin ligase activity; inflammation induced by E3 ubiquitin ligase activity; inflammation induced by TNF activity; apoptosis in activated T cells; angiogenesis; uncontrolled cell proliferation; uncontrolled cell proliferation mediated by E3 ubiquitin ligase activity; and translation of Hepatitis C-encoded proteins. Methods for measuring these activities and processes are found in U.S. patent application Ser. Nos. 10/039,761; 09/062,330; 09/293,670; 09/826,312; 09/050,861; 09/425,324; 09/076,624, each incorporated herein in their entirety by reference; and U.S. Provisional Patent Application Serial No. 60/316,723, incorporated herein in its entirety by reference.

In one embodiment, the present invention is useful in identifying modulators of the immune response. For example, activation of B-cells initiates various facets of humoral immunity, including immunoglobulin synthesis and antigen presentation by B-cells. Activation is mediated by engagement of the B-cell receptor (BCR), for example by binding of anti-IgM F(ab′) fragments, which induces several signal transduction pathways leading to various responses by the B-cell, including immunoglobulin synthesis and secretion, apoptosis, expression of cell surface marker CD69, and modulation of IgH promoter activity. cDNA expression vector are introduced into appropriate B-cell lines, such as Ramos Human B-cell lines, M12.4 etc., to identify various effectors of the signaling pathways activated by B-cell receptor engagement. The assays may comprise determining the level of CD69 cell surface marker (i.e. by fluorescently labeled anti-CD69 antibody and FACS selection of cells expressing high levels of CD69) following receptor activation.

In a preferred embodiment, the present methods and compositions are useful for screening for agents capable of modulating exocytosis. By “alteration” or “modulation” in relation to exocytosis is meant a decrease or increase in amount or frequency of exocytosis in one cell compared to another cell or in the same cell under different conditions. Often mediated by specialized cells, exocytosis is vital for a variety of cellular processes, including neurotramitter release by neurons, hormone release by adrenal chromaffin cells (adrenaline) and pancreatic β-cells (insulin), and histamine release by mast cells.

Disorders involving exocytosis are numerous. For example, inflammatory immune response mediated by mast cells leads to a variety of disorders, including asthma and allergies. Therapy for allergy remains limited to blocking mediators released by mast cells (i.e. anti-histamines) and non-specific anti-inflammatory agents, such as steroids and mast cell stabilizers. These treatments are only marginally effective in alleviating the symptoms of allergy. To identify cellular targets for drug design or candidate effectors of exocytosis, cDNA expression vectors may be introduced into appropriate cells, for example mast cells, and selected for modulation of exocytosis by assaying for changes in cellular exocytosis properties. These cells are stimulated with appropriate inducer if exocytosis is triggered by an inducing signal.

Assays for changes in exocytosis may comprise sorting cells in a fluorescence cell sorter (FACS) by measuring alterations of various exocytosis indicators, such as light scattering, fluorescent dye uptake, fluorescent dye release, granule release, and quantity of granule specific proteins (as provided in U.S. Ser. No 09/293,670, incorporated herein by reference). Use of combinations of indicators reduces background and increases specificity of the sorting assay.

The exocytosis assay based on changes in the cell's light scattering properties, including use of forward and side scatter properties of the cells, are indicative of the size, shape, and granule content of the cell. Multiparameter FACS selection based on light scattering properties of cells are well known in the art, (see Perretti, M. et al. (1990) J. Pharmacol. Methods 23: 187-94; Hide, I. et al. (1993) J. Cell Biol. 123: 585-93).

Assays based on uptake of fluorescent dyes reflect the coupling of exocytosis and endocytosis in which endocytosis levels indirectly reflect exocytosis levels since the cell attempts to maintain cell volume and membrane integrity as the amount of cell membrane rapidly changes when secretory vesicles fuse with the cell membrane. Preferred fluorescent dyes include styryl dyes, such as FM1-43, FM4-64, FM14-68, FM2-10, FM4-84, FM1-84, FM14-27, FM14-29, FM3-25, FM3-14, FM5-55, RH414, FM6-55, FM10-75, FM1-81, FM9-49, FM4-95, FM4-59, FM9-40, and combinations thereof. Styryl dyes such as FM 1-43 are only weakly fluorescent in water but very fluorescent when associated with a membrane, such that dye uptake by endocytosis is readily discernable (Betz, et al. (1996) Current Opinion in Neurobiology, 6:365-371; Molecular Probes, Inc., Eugene, Oreg., “Handbook of Fluorescent Probes and Research Chemicals”, 6th Edition, 1996, particularly, Chapter 17, and more particularly, Section 2 of Chapter 17, (including referenced related chapter), hereby incorporated herein by reference). Useful solution dye concentration is about 25 to 1000-5000 nM, with from about 50 to about 1000 nM being preferred, and from about 50 to 250 being particularly preferred.

Exocytosis assays based on fluorescent dye release rely on release of dye that is taken up passively by the cell or dye that is actively endocytosed by the cell. Release of dyes initially taken up by a cell results in decreased cellular fluorescence and presence of the dye in the cellular medium, thus providing two ways to measure dye release. For example, styryl dyes taken up into cells by endocytosis is released into the cellular media by exocytosis, resulting in decreased cellular fluorescence and presence of the dye in the medium. Another dye release assay uses low pH dyes, such as acridine orange, LYSOTRACKER™ red, LYSOTRACKER™ green, and LYSOTRACKER™ blue (Molecular Probes, supra), which stains exocytic granules when dye is internalized by the cell.

Preferential staining of exocytic granules when the vesicles fuse with the cell membrane provides an additional assay for measuring exocytosis. Annexin V, which binds to phospholipid (phospahtidyl serine) in a divalent ion dependent manner, specifically binds to exocytic granules present on the cell surface but fails to bind internally localized exocytic granules. This property of Annexin provides a basis for determining exocytosis by the level of Annexin bound to cells. Cells show an increase in Annexin binding in proportion to the time and intensity of the exocytic response. Annexin is detectable directly by use of fluorescently labeled Annexin derivatives (i.e. FITC, TRITC, AMCA, APC, or Cy-5 fluorescent labels), or indirectly by use of Annexin modified with a primary label (e.g. biotin), which is detected using a labeled secondary agent that binds to the primary label (e.g. fluorescently labeled avidin).

Alternatively, in a preferred embodiment the exocytosis indicators are engineered into the cells. For example, recombinant proteins comprising fusion proteins of a granule specific, or a secreted protein, and a reporter molecule are expressed in a cell by transforming the cells with a fusion nucleic acid encoding a fusion protein comprising a granule specific or secreted protein and a reporter protein. This is generally done as is known in the art, and will depend on the cell type. Generally, for mammalian cells, retroviral vectors are preferred for delivery of the fusion nucleic acid. Preferred reporter molecules include, but are not limited to, Aequoria victoria GFP, Renilla mulleris GFP, Renilla reniformis GFP, Renilla ptilosarcus, GFP, BFP, YFP, and enzymes including luciferases (Renilla, firefly etc.) and β-galactosidases. Presence of the granule protein-reporter fusion construct on the cell surface or presence of secreted protein-reporter fusion construct in the medium indicates the level of exocytosis in the cells. Thus, in one preferred embodiment cells are transformed with retroviral vectors expressing a fusion protein comprising granule specific (i.e. secretory vesicle) proteins, such as VAMP (synaptobrevin) or synaptotagmin, fused to a GFP reporter molecule. The cells are monitored for localization of the fusion protein to the cell membrane. Candidate agents (cDNA expression vectors) are introduced into these transformed cells and are tested for their ability to affect distribution of the fusion protein. Since the definition of granule specific proteins encompasses mediators released during exocytosis, including, but not limited to, serotonin, histamine, heparin, hormones, etc., these granule proteins may be identified using specific antibodies.

In a preferred embodiment, the present methods are useful in cancer applications. The ability to rapidly and specifically kill tumor cells is a cornerstone of cancer chemotherapy. In general, using the methods of the present invention, cDNA expression libraries can be introduced into any tumor cell (primary or cultured), and bioactive agents identified which by themselves induce apoptosis, cell death, loss of cell division or decreased cell growth. The methods of the present invention can be combined with other cancer therapeutics (e.g. drugs or radiation) to sensitize the cells and thus induce rapid and specific apoptosis, cell death, loss of cell division or decreased cell growth after exposure to a secondary agent. Similarly, the present methods may be used in conjunction with known cancer therapeutics to screen for agonists to make the therapeutic more effective or less toxic. This is particularly preferred when the chemotherapeutic is very expensive to produce such as taxol.

Known oncogenes such as v-Abl, v-Src, v-Ras, and others, induce a transformed phenotype leading to abnormal cell growth when transfected into certain cells. This is also a major problem with micro_metastases. Thus, in a preferred embodiment, non-transformed cells can be transfected with these oncogenes, and then cDNA libraries introduced into these cells, to select for bioactive agents which reverse or correct the transformed state. One of the signal features of oncogene transformation of cells is the loss of contact inhibition and the ability to grow in soft-agar. When transforming viruses are constructed containing v-Abl, v-Src, or v-Ras in IRES-puro retroviral vectors, infected into target 3T3 cells, and subjected to puromycin selection, all of the 3T3 cells hyper-transform and detach from the plate. The cells may be removed by washing with fresh medium. This can serve as the basis of a screen, since cells which express a bioactive agent will remain attached to the plate and form colonies.

Similarly, the growth and/or spread of certain tumor types is enhanced by stimulatory responses from growth factors and cytokines (PDGF, EGF, Heregulin, and others) which bind to receptors on the surfaces of specific tumors. In a preferred embodiment, the methods of the invention are used to inhibit or stop tumor growth and/or spread, by finding bioactive agents capable of blocking the ability of the growth factor or cytokine to stimulate the tumor cell. The methods involve the introduction of cDNA libraries into specific tumor cells with the addition of the growth factor or cytokine, followed by selection of bioactive agents which block the binding, signaling, phenotypic and/or functional responses of these tumor cells to the growth factor or cytokine in question.

Similarly, the spread of cancer cells (invasion and metastasis) is a significant problem limiting the success of cancer therapies. The ability to inhibit the invasion and/or migration of specific tumor cells would be a significant advance in the therapy of cancer. Tumor cells known to have a high metastatic potential (for example, melanoma, lung cell carcinoma, breast and ovarian carcinoma) can have cDNA expression libraries introduced into them, and peptides selected which in a migration or invasion assay, inhibit the migration and/or invasion of specific tumor cells. Particular applications for inhibition of the metastatic phenotype, which could allow a more specific inhibition of metastasis, include the metastasis suppressor gene NM23, which codes for a dinucleoside diphosphate kinase. Thus intracellular peptide activators of this gene could block metastasis, and a screen for its upregulation (by fusing it to a reporter gene) would be of interest. Many oncogenes also enhance metastasis. Peptides which inactivate or counteract mutated RAS oncogenes, v-MOS, v-RAF, A-RAF, v-SRC, v-FES, and v-FMS would also act as anti-metastatics. Peptides which act intracellularly to block the release of combinations of proteases required for invasion, such as the matrix metalloproteases and urokinase, could also be effective antimetastatics.

In a preferred embodiment, the libraries of the present invention are introduced into tumor cells known to have inactivated tumor suppressor genes, and successful reversal by either reactivation or compensation of the knockout would be screened by restoration of the normal phenotype. A major example is the reversal of p53-inactivating mutations, which are present in 50% or more of all cancers. Since p53's actions are complex and involve its action as a transcription factor, there are probably numerous potential ways a peptide or small molecule derived from a peptide could reverse the mutation. One example would be upregulation of the immediately downstream cyclin-dependent kinase p21 CIP1/WAF1. To be useful such reversal would have to work for many of the different known p53 mutations. This is currently being approached by gene therapy; one or more small molecules which do this might be preferable.

Another example involves screening for bioactive agents which restore the constitutive function of the brca-1 or brca-2 genes, and other tumor suppressor genes important in breast cancer such as the adenomatous polyposis coli gene (APC) and the homolog of the Drosophila discs-large gene (DIg), which are components of cell-cell junctions. Mutations of brca-1 are important in hereditary ovarian and breast cancers, and screening for bioactive agents capable of suppressing these cancers is an additional application of the present invention.

In a preferred embodiment, the methods of the present invention are used to create novel cell lines from cancers from patients. A retrovirally delivered candidate agents which inhibits the final common pathway of programmed cell death should allow for short- and possibly long-term cell lines to be established. Conditions of in vitro culture and infection of human leukemia cells will be established. There is a real need for methods which allow the maintenance of certain tumor cells in culture long enough to allow for physiological and pharmacological studies. Currently, some human cell lines have been established by the use of transforming agents such as Ebstein-Barr virus that considerably alters the existing physiology of the cell. On occasion, cells will grow on their own in culture but this is a random event. Programmed cell death (apoptosis) occurs via complex signaling pathways within cells that ultimately activate a final common pathway producing characteristic changes in the cell leading to a non-inflammatory destruction of the cell. It is well known that tumor cells have a high apoptotic index, or propensity to enter apoptosis in vivo. When cells are placed in culture, the in vivo stimuli for malignant cell growth are removed and cells readily undergo apoptosis. The objective would be to develop the technology to establish cell lines from any number of primary tumor cells, for example primary human leukemia cells, in a reproducible manner without altering the native configuration of the signaling pathways in these cells. By introducing nucleic acids encoding peptides which inhibit apoptosis, increased cell survival in vitro, and hence the opportunity to study signalling transduction pathways in primary human tumor cells, is accomplished. In addition, these methods may be used for culturing primary cells, i.e. non-tumor cells.

In a preferred embodiment, the present methods are useful in cardiovascular applications. In a preferred embodiment, cardiomyocytes may be screened for the prevention of cell damage or death in the presence of normally injurious conditions, including, but not limited to, the presence of toxic drugs (particularly chemotherapeutic drugs), for example, to prevent heart failure following treatment with adriamycin; anoxia, for example in the setting of coronary artery occlusion; and autoimmune cellular damage by attack from activated lymphoid cells (for example as seen in post viral myocarditis and lupus). Candidate bioactive agents are inserted into cardiomyocytes, the cells are subjected to the insult, and bioactive agents are selected that prevent any or all of: apoptosis; membrane depolarization (i.e. decrease arrythmogenic potential of insult); cell swelling; or leakage of specific intracellular ions, second messengers and activating molecules (for example, arachidonic acid and/or lysophosphatidic acid).

In a preferred embodiment, the present methods are used to screen for diminished arrhythmia potential in cardiomyocytes. The screens comprise the introduction of the candidate nucleic acids encoding candidate bioactive agents, followed by the application of arrythmogenic insults, with screening for bioactive agents that block specific depolarization of cell membrane. This may be detected using patch clamps, or via fluorescence techniques). Similarly, channel activity (for example, potassium and chloride channels) in cardiomyocytes could be regulated using the present methods in order to enhance contractility and prevent or diminish arrhythmias.

In a preferred embodiment, the present methods are used to screen for enhanced contractile properties of cardiomyocytes and diminish heart failure potential. The introduction of the libraries of the invention followed by measuring the rate of change of myosin polymerization/depolymerization using fluorescent techniques can be done. Bioactive agents which increase the rate of change of this phenomenon can result in a greater contractile response of the entire myocardium, similar to the effect seen with digitalis.

In a preferred embodiment, the present methods are useful to identify agents that will regulate the intracellular and sarcolemmal calcium cycling in cardiomyocytes in order to prevent arrhythmias. Bioactive agents are selected that regulate sodium-calcium exchange, sodium proton pump function, and regulation of calcium-ATPase activity.

In a preferred embodiment, the present methods are useful to identify agents that diminish embolic phenomena in arteries and arterioles leading to strokes (and other occlusive events leading to kidney failure and limb ischemia) and angina precipitating a myocardial infarct are selected. For example, bioactive agents which will diminish the adhesion of platelets and leukocytes, and thus diminish the occlusion events. Adhesion in this setting can be inhibited by the libraries of the invention being inserted into endothelial cells (quiescent cells, or activated by cytokines, i.e. IL-1, and growth factors, i.e. PDGF/EGF) and then screening for peptides that either: 1) downregulate adhesion molecule expression on the surface of the endothelial cells (binding assay); 2) block adhesion molecule activation on the surface of these cells (signaling assay); or 3) release in an autocrine manner peptides that block receptor binding to the cognate receptor on the adhering cell.

Embolic phenomena can also be addressed by activating proteolytic enzymes on the cell surfaces of endothelial cells, and thus releasing active enzyme which can digest blood clots. Thus, delivery of the libraries of the invention to endothelial cells is done, followed by standard fluorogenic assays, which will allow monitoring of proteolytic activity on the cell surface towards a known substrate. Bioactive agents can then be selected which activate specific enzymes towards specific substrates.

In a preferred embodiment, arterial inflammation in the setting of vasculitis and post-infarction can be regulated by decreasing the chemotactic responses of leukocytes and mononuclear leukocytes. This can be accomplished by blocking chemotactic receptors and their responding pathways on these cells. Candidate bioactive libraries can be inserted into these cells, and the chemotactic response to diverse chemokines (for example, to the IL-8 family of chemokines, RANTES) inhibited in cell migration assays.

In a preferred embodiment, arterial restenosis following coronary angioplasty can be controlled by regulating the proliferation of vascular intimal cells and capillary and/or arterial endothelial cells. Candidate bioactive agent libraries can be inserted into these cell types and their proliferation in response to specific stimuli monitored. One application may be intracellular peptides which block the expression or function of c_myc and other oncogenes in smooth muscle cells to stop their proliferation. A second application may involve the expression of libraries in vascular smooth muscle cells to selectively induce their apoptosis. Application of small molecules derived from these peptides may require targeted drug delivery; this is available with stents, hydrogel coatings, and infusion_based catheter systems. Peptides which downregulate endothelin_{—}1A receptors or which block the release of the potent vasoconstrictor and vascular smooth muscle cell mitogen endothelin-1 may also be candidates for therapeutics. Peptides can be isolated from these libraries which inhibit growth of these cells, or which prevent the adhesion of other cells in the circulation known to release autocrine growth factors, such as platelets (PDGF) and mononuclear leukocytes.

The control of capillary and blood vessel growth is an important goal in order to promote increased blood flow to ischemic areas (growth), or to cut-off the blood supply (angiogenesis inhibition) of tumors. Candidate bioactive agent libraries can be inserted into capillary endothelial cells and their growth monitored. Stimuli such as low oxygen tension and varying degrees of angiogenic factors can regulate the responses, and peptides isolated that produce the appropriate phenotype. Screening for antagonism of vascular endothelial cell growth factor, important in angiogenesis, would also be useful.

In a preferred embodiment, the present methods are useful in screening for decreases in atherosclerosis producing mechanisms to find peptides that regulate LDL and HDL metabolism. Candidate libraries can be inserted into the appropriate cells (including hepatocytes, mononuclear leukocytes, endothelial cells) and peptides selected which lead to a decreased release of LDL or diminished synthesis of LDL, or conversely to an increased release of HDL or enhanced synthesis of HDL. Bioactive agents can also be isolated from candidate libraries which decrease the production of oxidized LDL, which has been implicated in atherosclerosis and isolated from atherosclerotic lesions. This could occur by decreasing its expression, activating reducing systems or enzymes, or blocking the activity or production of enzymes implicated in production of oxidized LDL, such as 15 lipoxygenase in macrophages.

In a preferred embodiment, the present methods are used in screens to regulate obesity via the control of food intake mechanisms or diminishing the responses of receptor signaling pathways that regulate metabolism. Bioactive agents that regulate or inhibit the responses of neuropeptide Y (NPY), cholecystokinin and galanin receptors, are particularly desirable. Candidate libraries can be inserted into cells that have these receptors cloned into them, and inhibitory peptides selected that are secreted in an autocrine manner that block the signaling responses to galanin and NPY. In a similar manner, peptides can be found that regulate the leptin receptor.

In a preferred embodiment, the present methods are useful in neurobiology applications. Candidate libraries may be used for screening for anti_apoptotics for preservation of neuronal function and prevention of neuronal death. Initial screens would be done in cell culture. One application would include prevention of neuronal death, by apoptosis, in cerebral ischemia resulting from stroke. Apoptosis is known to be blocked by neuronal apoptosis inhibitory protein (NAIP); screens for its upregulation, or effecting any coupled step could yield peptides which selectively block neuronal apoptosis. Other applications include neurodegenerative diseases such as Alzheimer's disease and Huntington's disease.

In a preferred embodiment, the present methods are useful in bone biology applications. Osteoclasts are known to play a key role in bone remodeling by breaking down “old” bone, so that osteoblasts can lay down “new” bone. In osteoporosis one has an imbalance of this process. Osteoclast overactivity can be regulated by inserting candidate libraries into these cells, and then looking for bioactive agents that produce: 1) a diminished processing of collagen by these cells; 2) decreased pit formation on bone chips; and 3) decreased release of calcium from bone fragments.

The present methods may also be used to screen for agonists of bone morphogenic proteins, hormone mimetics to stimulate, regulate, or enhance new bone formation (in a manner similar to parathyroid hormone and calcitonin, for example). These have use in osteoporosis, for poorly healing fractures, and to accelerate the rate of healing of new fractures. Furthermore, cell lines of connective tissue origin can be treated with candidate libraries and screened for their growth, proliferation, collagen stimulating activity, and/or proline incorporating ability on the target osteoblasts. Alternatively, candidate libraries can be expressed directly in osteoblasts or chondrocytes and screened for increased production of collagen or bone.

In a preferred embodiment, the present methods are useful in skin biology applications. Keratinocyte responses to a variety of stimuli may result in psoriasis, a proliferative change in these cells. Candidate libraries can be inserted into cells removed from active psoriatic plaques, and bioactive agents isolated which decrease the rate of growth of these cells.

In a preferred embodiment, the present methods are useful in the regulation or inhibition of keloid formation (i.e. excessive scarring). Candidate libraries inserted into skin connective tissue cells isolated from individuals with this condition, and bioactive agents isolated that decrease proliferation, collagen formation, or proline incorporation. Results from this work can be extended to treat the excessive scarring that also occurs in burn patients. If a common peptide motif is found in the context of the keloid work, then it can be used widely in a topical manner to diminish scarring post burn.

Similarly, wound healing for diabetic ulcers and other chronic “failure to heal” conditions in the skin and extremities can be regulated by providing additional growth signals to cells which populate the skin and dermal layers. Growth factor mimetics may in fact be very useful for this condition. Candidate libraries can be inserted into skin connective tissue cells, and bioactive agents isolated which promote the growth of these cells under “harsh” conditions, such as low oxygen tension, low pH, and the presence of inflammatory mediators.

Cosmeceutical applications of the present invention include the control of melanin production in skin melanocytes. A naturally occurring peptide, arbutin, is a tyrosine hydroxylase inhibitor, a key enzyme in the synthesis of melanin. Candidate libraries can be inserted into melanocytes and known stimuli that increase the synthesis of melanin applied to the cells. Bioactive agents can be isolated that inhibit the synthesis of melanin under these conditions.

In a preferred embodiment, the present methods are useful in endocrinology applications. The retroviral peptide library technology can be applied broadly to any endocrine, growth factor, cytokine or chemokine network which involves a signaling peptide or protein that acts in either an endocrine, paracrine or autocrine manner that binds or dimerizes a receptor and activates a signaling cascade that results in a known phenotypic or functional outcome. The methods are applied so as to isolate a peptide which either mimics the desired hormone (i.e., insulin, leptin, calcitonin, PDGF, EGF, EPO, GMCSF, IL1-17, mimetics) or inhibits its action by either blocking the release of the hormone, blocking its binding to a specific receptor or carrier protein (for example, CRF binding protein), or inhibiting the intracellular responses of the specific target cells to that hormone. Selection of peptides which increase the expression or release of hormones from the cells which normally produce them could have broad applications to conditions of hormonal deficiency.

In a preferred embodiment, the present methods are useful in infectious disease applications. Viral latency (herpes viruses such as CMV, EBV, HBV, and other viruses such as HIV, hepatitis c, hepatitis b, hepatitis a, influenza virus) and their reactivation are a significant problem, particularly in immunosuppressed patients (patients with AIDS and transplant patients). The ability to block the reactivation and spread of these viruses is an important goal. Cell lines known to harbor or be susceptible to latent viral infection can be infected with the specific virus, and then stimuli applied to these cells which have been shown to lead to reactivation and viral replication. This can be followed by measuring viral titers in the medium and scoring cells for phenotypic changes. Candidate libraries can then be inserted into these cells under the above conditions, and peptides isolated which block or diminish the growth and/or release of the virus. As with chemotherapeutics, these experiments can also be done with drugs which are only partially effective towards this outcome, and bioactive agents isolated which enhance the virucidal effect of these drugs.

One example of many is the ability to block HIV-1 infection. HIV-1 requires CD4 and a co-receptor which can be one of several seven transmembrane G-protein coupled receptors. In the case of the infection of macrophages, CCR-5 is the required co-receptor, and there is strong evidence that a block on CCR-5 will result in resistance to HIV-1 infection. There are two lines of evidence for this statement. First, it is known that the natural ligands for CCR-5, the CC chemokines RANTES, MIP1a and MIP1b are responsible for CD8+ mediated resistance to HIV. Second, individuals homozygous for a mutant allele of CCR-5 are completely resistant to HIV infection. Thus, an inhibitor of the CCR-5/HIV interaction would be of enormous interest to both biologists and clinicians. The extracellular anchored constructs offer superb tools for such a discovery. Into the transmembrane, epitope tagged, glycine-serine tethered constructs (ssTM V G20 E TM), one can place a random, cyclized peptide library of the general sequence CNNNNNNNNNNC or C—(X)ₙ—C. Then one infects a cell line that expresses CCR-5 with retroviruses containing this library. Using an antibody to CCR-5 one can use FACS to sort desired cells based on the binding of this antibody to the receptor. All cells which do not bind the antibody will be assumed contain inhibitors of this antibody binding site. These inhibitors, in the retroviral construct can be further assayed for their ability to inhibit HIV-1 entry.

Viruses are known to enter cells using specific receptors to bind to cells (for example, HIV uses CD4, coronavirus uses CD13, murine leukemia virus uses transport protein, and measles virus usesCD44) and to fuse with cells (HIV uses chemokine receptor). Candidate libraries can be inserted into target cells known to be permissive to these viruses, and bioactive agents isolated which block the ability of these viruses to bind and fuse with specific target cells.

In a preferred embodiment, the present invention finds use with infectious organisms. Intracellular organisms such as mycobacteria, listeria, salmonella, pneumocystis, yersinia, leishmania, T. cruzi, can persist and replicate within cells, and become active in immunosuppressed patients. There are currently drugs on the market and in development which are either only partially effective or ineffective against these organisms. Candidate libraries can be inserted into specific cells infected with these organisms (pre- or post-infection), and bioactive agents selected which promote the intracellular destruction of these organisms in a manner analogous to intracellular “antibiotic peptides” similar to magainins. In addition peptides can be selected which enhance the cidal properties of drugs already under investigation which have insufficient potency by themselves, but when combined with a specific peptide from a candidate library, are dramatically more potent through a synergistic mechanism. Finally, bioactive agents can be isolated which alter the metabolism of these intracellular organisms, in such a way as to terminate their intracellular life cycle by inhibiting a key organismal event.

Antibiotic drugs that are widely used have certain dose dependent, tissue specific toxicities. For example renal toxicity is seen with the use of gentamicin, tobramycin, and amphotericin; hepatotoxicity is seen with the use of INH and rifampin; bone marrow toxicity is seen with chloramphenicol; and platelet toxicity is seen with ticarcillin, etc. These toxicities limit their use. Candidate libraries can be introduced into the specific cell types where specific changes leading to cellular damage or apoptosis by the antibiotics are produced, and bioactive agents can be isolated that confer protection, when these cells are treated with these specific antibiotics.

Furthermore, the present invention finds use in screening for bioactive agents that block antibiotic transport mechanisms. The rapid secretion from the blood stream of certain antibiotics limits their usefulness. For example penicillins are rapidly secreted by certain transport mechanisms in the kidney and choroid plexus in the brain. Probenecid is known to block this transport and increase serum and tissue levels. Candidate agents can be inserted into specific cells derived from kidney cells and cells of the choroid plexus known to have active transport mechanisms for antibiotics. Bioactive agents can then be isolated which block the active transport of specific antibiotics and thus extend the serum half-life of these drugs.

Other agents which may be selected using the present invention include: 1) agents which block the activity of transcription factors, using cell lines with reporter genes; 2) agents which block the interaction of two known proteins in cells, using the absence of normal cellular functions, the mammalian two hybrid system or fluorescence resonance energy transfer mechanisms for detection; and 3) agents may be identified by tethering a random peptide to a protein binding region to allow interactions with molecules sterically close, i.e. within a signaling pathway, to localize the effects to a functional area of interest.

Once identified, the host cell targets or viral targets that are identified are screened for agents that inhibit or alter their activity. That is, the targets were initially identified because their inhibition resulted in inhibition of IRES activity. Thus, it is desirable to isolate other agents that also effectively inhibit the targets. Accordingly, the targets are screened against libraries of compounds as is known in the art to identify molecules that bind and/or inhibit the activity of the target. Alternatively, targets are screened for agents that increase their activity.

Libraries that can be screened include small molecule libraries, combinatorial libraries and the like. Preferably the library is a small molecule library. Small molecules offer the advantage that the molecules identified are frequently membrane permeable and provide effective lead compounds for the development of therapeutics. While the size of the molecules can vary significantly, in some embodiments it is preferred that the small molecules are less than about 10k.

Accordingly, the invention also provides a method of treating a viral infection or inhibiting a virus by contacting a virus infected cell with a compound identified by the above screens.

In an alternative embodiment the molecule identified that inhibited IRES activity may itself be a therapeutic molecule. In a preferred embodiment the molecule is a cyclic peptide identified in a screen for anti-IRES molecules as described herein. The cyclic peptide preferably is a therapeutic molecule. In an alternative embodiment the molecule is a linear peptide. When this occurs, in a particularly preferred embodiment the linear peptide sequence is made into a cyclic peptide because of the constrained structure of the cyclic peptide relative to the linear peptide. Without being bound by theory, it is thought that the constrained structure provides increased stability and increased interaction with the target molecule. Thus, the constrained structure is advantageous.

Alternatively, the cyclic peptide is not itself the therapeutic molecule, but it serves as a lead molecule for development of a therapeutic molecule. In this embodiment the lead cyclic peptide is modified and screened for increased bioactivity, i.e. increased anti-IRES activity. The cyclic peptide can be modified randomly. Alternatively, the cyclic peptide is modified in a rational basis. By “rational basis” is meant that the cyclic peptide is modified or mutated based on known interactions with the target molecule and/or to include modifications that increase its therapeutic effectiveness. Such modifications are based on knowledge gleaned from but not limited to structure function analysis, and structure analysis, such as NMR or x-ray crystallography.

Preferably the cyclic peptide serves as a model for the selection or synthesis of small molecules, i.e. organic molecules that are therapeutic molecules. In this embodiment the structure of the cyclic peptide is predicted or determined and small molecules are selected, screened for or synthesized that have similar structures. The small molecule need not have the precise structure as the cyclic peptide. Indeed, in many instances the small molecule may only be similar to a portion of the cyclic peptide. Preferably the small molecule resembles the portion of the cyclic peptide that interacts with the target molecule.

In addition, once a target molecule is identified that disrupts or alters IRES mediated expression of genes, the target molecule is validated in a cell system using an inducible promoter system. In this embodiment the target molecule is under the control of an inducible promoter. The phenotype of cells that have been induced for expression of the target molecule are compared with the phenotype of cells that have not been induces for expression of the target molecule. Additional disclosure regarding inducible expression systems is found in U.S. Ser. No. 10/096,339, filed Mar. 8, 2002, which is expressly incorporated herein by reference. An advantage in performing the validation is that it eliminates or reduces false positives. That is, following the initial screen with the library, the positive molecules are then screened in the validation assay. If the phenotype of the initial screen correlates with the phenotype in the validation assay, the positive molecule or bioactive agent is selected for further studies and optimization. Thus, the present invention provides a method of validating a positive molecule identified in a high-throughput screen by correlating the activity of the molecule in one assay system with the activity of the molecule in a different assay system.

The following examples serve to more fully describe the manner of using the above-described invention, as well as to set forth the best modes contemplated for carrying out various aspects of the invention. It is understood that these examples in no way serve to limit the true scope of this invention, but rather are presented for illustrative purposes. All references cited herein are incorporated by reference. CL EXAMPLES

Screening 5-mer Cyclic Neptide Library for HCV IRES Inhibitors

PhxA cells were seeded in 10 cm tissue culture dishes and cultured at 37° C., 5% CO₂ for 48 hours before transfection (yielding around 1×10⁷ cells). Plasmid construct of the cyclic peptide library was transfected into PhxA cells using the calcium phosphate co-precipitation method. Retrovirus-containing supernatant was collected 24 hours later for infection.

Screening cells (2×10⁶) were infected with retroviral cyclic peptide library using the spin-infection method. The infected screening cells were incubated at 37° C., 5% CO₂ for 3 days before bulk-sorted for BFP positive. The BFP-positive cells were further incubated at 37° C., 5% CO₂ for 3 more days and single-cell-cloned into 96-well plates. The cloning criteria was high BFP expression, high RFP expression and low GFP expression.

Single cells were cultured in 20% serum for 5 days for amplification. Then the cells in each well were equally split into medium without doxycycline (−DOX, resulting in the expression of cyclic peptide) and medium with doxycycline (+DOX, resulting in the cyclic peptide expression to be turned off). The cells were further cultured at 37° C., 5% CO₂ for 5 more days before corresponding −DOX and +DOX plates of the same clone were analyzed by FACS and hits were selected. The FACS criteria to define a positive hit was that the +DOX sample had a higher GFP geometric mean than the −DOX sample. If a cyclic peptide is an IRES inhibitor, addition of doxycycline will turn off the cyclic peptide expression and HCV IRES-dependent translation (reported by GFP) will be de-repressed compare to the −DOX sample and have a higher geometric mean.

Hits were selected and expanded for RNA-preparation. The RNA samples were used in RT-PCR reactions to rescue the DNA sequence coding for the cyclic peptides. After sequencing, the cyclic peptide coding DNA sequence were cloned into the library vector and made into retrovirus for transferring the cyclic peptide into a second round of infection to confirm the inhibition effect of the cyclic peptide.

We claim:

1. A method of assaying for a potential anti-HCV agent comprising:
a) providing cells comprising a nucleic acid construct comprising:
i) an HCV internal ribosome entry site (IRES); and
ii) a reporter gene;
b) contacting said cells with a library of nucleic acids, whereby said nucleic acids are expressed in said cells forming candidate agents;
c) screening said cells for altered expression of said reporter gene.

2. A method for assaying for anti-HCV agents comprising:
a) providing cells comprising the HCV genome;
b) contacting said cells with a library of nucleic acids, whereby said nucleic acids are expressed in said cells forming candidate agents; and
c) screening for cells exhibiting altered HCV production.

3. A method of assaying for a potential antiviral agent comprising:
a) providing cells comprising a nucleic acid construct comprising:
i) a viral internal ribosome entry site (IRES); and
ii) a reporter gene;
b) contacting said cells with a library of nucleic acids, whereby said nucleic acids are expressed in said cells forming candidate agents;
c) screening said cells for altered expression of said reporter gene.

4. The method according to claim 1, 2 or 3, wherein said library of nucleic acids is introduced into the cells with a vector.

5. The method according to claim 4, wherein said vector is a viral vector.

6. The method according to claim 5, wherein said viral vector is selected from the group consisting of retrovirus, adenovirus, AAV, and lentivirus

7. The method according to claim 6, wherein said viral vector is retrovirus.

8. The method according to claim 1, 2 or 3, wherein said library encodes peptides.

9. The method according to claim 8, wherein said peptides are random peptides.

10. The method according to claim 9, wherein said peptides are cyclic peptides.

11. The method according to claim 1, 2 or 3, wherein said library is a cDNA library.

12. The method according to claim 11, wherein said cDNA library is a randomly fragmented cDNA library.

13. The method according to claim 1, 2 or 3, wherein said library of nucleic acids encodes anti-sense molecules.

14. The method according to claim 13, further comprising identifying the wild-type nucleic acid complementary to the antisense nucleic acid expressed in cells exhibiting an altered phenotype.

15. The method according to claim 1, 2 or 3, wherein said library encodes dominant negative cellular polypeptides, or fragments thereof.

16. The method according to claim 15, further comprising identifying the wild-type nucleic acid corresponding to the cDNA expressed in cells exhibiting an altered phenotype.

17. The method according to claim 1, 2 or 3, wherein the nucleic acid library encodes fusion polypeptides.

18. The method according to claim 17, wherein said fusion polypeptides comprise said candidate agent and a presentation structure capable of presenting said targeting domain in a conformationally restricted form.

19. The method according to claim 17, wherein said fusion polypeptides comprise a candidate agent domain and a reporter domain.

20. The method according to claim 19, wherein said reporter domain comprises a reporter selected from the group consisting of a GFP and a luciferase.

21. The method according to claim 20, wherein said GFP is an Aequorea GFP or a Renilla GFP.

22. The method according to claim 21, wherein said GFP is Renilla mulleri GFP.

23. The method according to claim 22, wherein said reporter gene comprising Renilla mulleri GFP is codon-optimized for expression in eukaryotic cells.

24. The method according to claim 1, 2 or 3, further comprising;
d) identifying the nucleic acid that encodes said candidate agent.

25. The method according to claim 24, further comprising:
d) identifying a cellular polypeptide that binds said candidate agent.

26. The method according to claim 25, wherein the cellular polypeptide that binds to said candidate agent is identified by a method selected from the group consisting of a two hybrid screen and affinity chromatography.

27 The method according to claim 25, further comprising:
e) screening a library of small molecules for at least one molecule that binds to said candidate agent or cellular polypeptide.

28 The method according to claim 27, further comprising identifying said molecule that binds to said candidate agent or cellular polypeptide.
